(19) United States
(12) Patent Application Publication
(10) Pub. No.: US 2007/0166281 A1
(43) Pub. Date: Jul. 19, 2007

(54) Chloroquine coupled antibodies and other proteins with methods for their synthesis

(76) Inventor: Kenneth M. Kosak, West Valley City, UT (US)

Correspondence Address: Kenneth M. Kosak, 3194 South 4400 West, West Valley City, UT, 84120, US

(21) Appl. No.: 11/709,965

(22) Filed: Feb. 22, 2007

Related U.S. Application Data
(63) Continuation-in-part of application No. 11/360,111, filed on Feb. 22, 2006.
Application No. 11/360,111 is a continuation-in-part of application No. 11/323,389, filed on Dec. 29, 2005, now abandoned.
Application No. 11/323,389 is a continuation-in-part of application No. PCT/US05/33310, filed on Sep. 15, 2005.
Application No. PCT/US05/33310 is a continuation-in-part of application No. 10/923,112, filed on Aug. 21, 2004.

(51) Int. Cl. A61K 39/395; A61K 38/19; A61K 38/20; A61K 38/21; A61K 31/675; A61K 38/28; A61K 31/4706; A61K 31/4709

(52) U.S. Cl. 424/85.1; 424/178.1; 514/3; 514/12; 514/313; 424/85.4; 424/85.2; 514/80

(57) ABSTRACT

This invention discloses compositions of chloroquine-coupled active agents such as therapeutic antibodies or insulin, including methods for their preparation. The prior art has shown that chloroquines given as free drug in high enough concentration, enhances the release of various agents from cellular endosomes into the cytoplasm. The purpose of these compositions is to provide a controlled amount of chloroquine at the same site where the drug is delivered, thereby reducing the overall dosage needed. The compositions comprise a chloroquine substance coupled to a drug directly or through a variety of pharmaceutical carrier substances. The carrier substances include polysaccharides, synthetic polymers, proteins, micelles and other substances for carrying and releasing the chloroquine compositions in the body for therapeutic effect. The compositions can also include a biocleavable linkage for carrying and releasing the drug for therapeutic or other medical uses. The invention also discloses carrier compositions that are coupled to targeting molecules for targeting the delivery of chloroquine substances and antibody or insulin to their site of action.

### RELATED PATENT APPLICATIONS

This is a continuation-in-part application of U.S. patent application Ser. No. 11/360,111, filed Feb. 22, 2006, which is a CIP of U.S. patent application Ser. No. 11/323,389, filed Dec. 29, 2005, which is a CIP of PCT application No.PCT/US2005/033310, filed Sep. 15, 2005, which is a CIP of U.S. patent application Ser. No. 10/923,112, filed Aug. 21, 2004. The entire contents of these applications are incorporated herein.

### TECHNICAL FIELD OF THE INVENTION

This invention discloses new chloroquine compositions comprising chloroquine substances (chloroquines) coupled to antibody drugs and chloroquine substances coupled to other proteins and peptides for pharmaceutical, agricultural, diagnostic and research use. These chloroquine substances include covalent and noncovalent linkages coupling antibody substances, protein or peptide active agents with chloroquines or chloroquine substances, defined herein.

The composition can also include various carrier substances to which both the chloroquines and antibodies or other protein or peptides are coupled to produce a carrier composition (carrier). The carrier substances include polysaccharides, synthetic polymers, proteins, peptides and other substances for carrying and releasing the chloroquine compositions into the body for therapeutic effect.

Preferred carrier compositions contain biocleavable linkages that release the active agents and chloroquines under controlled conditions. The carrier compositions can also include targeting molecules for delivery of active agents and chloroquines to their desired site of action. The invention also discloses methods for preparing said compositions.

### BACKGROUND OF THE PRIOR ART

Therapeutic proteins and peptides including antibodies (protein drugs) used alone or as targeting molecules coupled to various active agents, are used in many disease therapies. Therapeutic antibodies and other proteins taken into target cells frequently suffer from degradation due to cellular endosomes and/or lysosomes.

It is well known in the prior art that “lysosomotropic” agents such as chloroquines are useful in releasing substances from lysosomes to avoid degradation. Chloroquines are known to improve DNA transfections and R. Marches, et al, Int J Cancer 112, 492-501 (2004) showed that chloroquine increases the anti tumor activity of the antibody drug Herceptin™. Chloroquine is also an “insulin-sparing” agent that can reduce the need for injected insulin by 30% for diabetic patients (A. Quatraro, et al, Ann Intern Med. 112, 678-81 (1990)). However, there is no disclosure of coupling chloroquines to protein, peptides, antibody or insulin.

It is also well known that chloroquines are synergistic with other active agents against many infectious diseases and certain cancer cells. S. T. Donta in Medical Sci. Monitor 9, 136-142 (2003) reported that hydroxychloroquine in combination with certain macrolide drugs improved the treatment of lyme disease over the use of macrolides alone. However, all such treatments involve dosing the patient with free chloroquines and there is no disclosure or suggestion of coupling chloroquines to the active agents to improve the synergistic effects.

There are several U.S. patents disclosing chloroquine for use against a variety of diseases either alone or in combination with other drugs. For instance, U.S. Pat. No. 4,181,725 and A. M. Krieg, et al, U.S. Patent Applic. 20040009949 disclose the use of chloroquine for treating various autoimmune diseases in combination with inhibitory nucleic acids. Also of interest are U.S. Pat. Nos. 5,736,557 and 6,417,177 where several chloroquine derivatives are disclosed. However, nothing in the prior art discloses or suggests the chloroquine-coupled compositions claimed in the present invention.

This may be due to reports in the art of nucleic acids that teach away from its in vivo use due to chloroquine toxicity. For instance, J. M. Benns, et al, recently reported, “Although chloroquine has proven to aid in the release of the plasmid DNA into the cytoplasm, it has been found to be toxic and thus cannot be used in vivo.” (1^{st} paragraph, Bioconj. Chem. 11, 637-645, (2000). This problem is partly due to the fact that relatively high concentrations of free chloroquine are needed to reach the same site as the nucleic acid in the endosome.

In the prior art of drug treatment, another serious problem is that drug-resistant strains of viruses (i.e. HIV) and other pathogens are rapidly increasing. Combination drug therapies have been proven more effective than single drugs against several diseases including cancer.

There are now several fixed-dose-combination (FDC) treatments comprising mixtures of two or more “free” drugs in one capsule. However, resistant strains have still developed even against such combinations of free drugs. One of the key problems is the variation in pharmacokinetics. Each free drug administered in a mixture quickly separates by dilution from a dissolved oral capsule or even when injected into the bloodstream.

These separated drugs can then vary widely in uptake, distribution and metabolism. Because of their different behaviors, the drugs may not get to the same infected cells at the same time or in the desired concentrations to give optimal synergistic effect.

Surprisingly, it was found that the embodiments of the present invention solve several problems by covalently coupling one or more chloroquine moieties directly to the therapeutic protein, peptide or antibody so that the chloroquine and protein drug are taken together to the same site. Therefore, every moiety of antibody is automatically associated with the required amount of chloroquine. There is no longer any need to use excess chloroquine because the compositions of the present invention automatically provide the benefits of chloroquine treatment at the same site as the antibody substance. It will be apparent that the compositions of the instant invention provide other unexpected advantages such as stability, cost savings and simple synthesis methods.

### SUMMARY DISCLOSURE OF THE INVENTION

The prior art has shown that chloroquines given as free drug in high enough concentration, enhance the efficacy and transport of various agents including antibodies from cellular endosomes into the cytoplasm. The purpose of this invention is to provide a controlled amount of chloroquine substance at the same therapeutic site as protein drug such as antibody, thereby reducing the overall chloroquine dosage needed.

The present invention is a chloroquine composition comprised of any suitable chloroquine substance coupled to a protein drug such as an antibody or antibody substance defined herein. Optionally, one or several moieties can also be coupled to the protein drug such as active agents, targeting molecules and transduction vectors disclosed herein to provide other desirable properties. The composition can also include various carrier substances to which both the chloroquine and protein drug are coupled to produce a carrier composition.

The carrier substances of this invention are divided into categories of suitable substances that include proteins, carbohydrates, polymers, grafted polymers and amphiphilic molecules as disclosed herein. The carrier composition can include a biodegradable linkage between the chloroquines and the carrier substance and/or between the protein drug and the carrier substance to provide controlled release of the chloroquines and/or th protein or peptide active agent or antibody after the carrier has reached its site of action. Optionally, one or several moieties can also be coupled to the carrier such as targeting molecules and transduction vectors disclosed herein.

Any suitable synthesis method now used for preparing polymers conjugated to various moieties, with suitable modification, is applicable to the synthesis of this invention. A distinguishing property of this invention is that the chloroquines and protein drug are conjugated.

For use as carriers, suitable polymers such as dextran or polyethylene glycol (PEG) are commercially available in a variety of molecular masses. Based on their molecular size, they are arbitrarily classified into low molecular weight (Mw<20,000) and high molecular weight (Mw>20,000). In this invention, polymers carriers of a molecular weight of 20,000 or greater are preferred when the purpose is to prevent rapid elimination due to renal clearance. The instant invention thereby provides new properties and unexpected advantages.

It will be understood in the art of protein drugs, antibodies, nucleic acids and other active agents, that there are limitations as to which derivatives, coupling agents or other substances can be used with chloroquines to fulfill their intended function. The terms “suitable” and “appropriate” refer to substances or synthesis methods known to those skilled in the art that are needed to perform the described reaction or to fulfill the intended function. It will also be understood in the art of chloroquines, active agents, antibody substances and drug carriers that there are many substances defined herein that, under specific conditions, can fulfill more than one function. Therefore, if they are listed or defined in more than one category, it is understood that each definition or limitation depends upon the conditions of their intended use.

### Industrial Applicability and Use

These compositions containing chloroquine substances are for the pharmaceutical, agricultural and research markets. The compositions are intended to improve the treatment of disease and other therapeutic applications in humans, other animals and plants. Many antibody substances and other protein drugs can be made more effective through the combinative effects of chloroquine substances. The compositions of this invention are useful for administration to people or other animals in a suitable dosage regimen by any suitable route such as orally; by any injection route (i.e. intravenous, subcutaneous, intramuscular, intracranial, etc.); by pulmonary, nasal, anal, vaginal or urethral route; through the eye, ear, nose or throat and topically through the skin. Administration can include the use of any suitable drug delivery device, composition or vehicle that facilitates delivery of the compositions of this invention into the body.

### Best Modes For Carrying out the Invention

For the purposes of disclosing this invention, certain words, phrases and terms used herein are defined below. Wherein certain definitions comprise a list of substances preceded by any grammatical form of the term “includes”, such substances are presented as examples taken from a group of substances known in the art to fit the said definition and the invention is not limited to the examples and references given. All references listed herein, and references therein, are incorporated into this invention by reference, including active agents, chloroquine substances, protein drugs, antibody substances, nucleic acid sequences, peptide sequences and methods for their synthesis or use.

### Chloroquine Substance

A chloroquine substance, is defined here as a usually (but not necessarily), lysosomotropic substance that includes, but is not limited to, quinoline and quinoline derivatives and quinoline compounds, especially 4-aminoquinoline and 2-phenylquinoline compounds and amino, thio, phenyl, alkyl, vinyl and halogen derivatives thereof. The most preferred chloroquine substances (sometimes called “chloroquines”), include chloroquine, hydroxychloroquines, amodiaquins (camoquines), amopyroquines, halofantrines, mefloquines; nivaquines, primaquines, tafenoquine and quinone imines and chloroquine analogs or derivatives wherein the (−)-enantiomers of chloroquine and hydroxychloroquine are most preferred. Preferred chloroquine substances listed below with their chemical name, include but are not limited to: 7-chloro-4-(4-diethylamino-1-methylbutylamino)quinoline (chloroquine); 7-chloro-4-(4-ethyl-(2-hydroxyethyl)-amino-1-methylbutylamino)quinoline (hydroxychloroquine); 7-fluoro-4-(4-diethylamino-1-methylbutylamino)quinoline; 4-(4-diethylamino-1-methylbutylamino) quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino) quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxyethyl)-amino-1-methylbutylamino-)quinoline7-hydroxy-4-(4-ethyl-(2-hydroxyethyl)-amino-1-methylbutylamino) quinoline; hydroxychloroquine phosphate; 7-chloro-4-(4-ethyl-(2-hydroxyethy-1)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 3-fluoro-4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof.

Preferred chloroquine substances include the agents, analogs and derivatives disclosed by D. J. Naisbitt, et al, in J. Pharmacol. Exp. Therapy 280, 884-893 (1997), and any quinolin-4-yl derivatives including N,N′-bis(quinolin-4-yl) derivatives disclosed in U.S. Pat. No. 5,736,557 and in references in the foregoing which are incorporated herein.

### Activated Chloroquine Substance

An activated chloroquine substance is defined for this invention as a chloroquine substance suitably derivatized to contain or coupled to, an active coupling group that is capable of coupling to a functional group on any suitable moiety such as an antibody or carrier substance, defined herein. Preferred embodiments in this invention include, but are not limited to, chloroquine substances that contain active aldehydes, anhydrides, peroxides, N-hydroxysuccinimide esters, 3-nitrophenyl esters, imidoesters, maleimides, mustards and S-ethyl esters, among others.

### Protein or Peptide Active Agents

Protein active agents or peptide active agents are defined here as limited to pharmaceutical proteins, including antibodies, and peptides that are stimulatory, inhibitory, antimetabolic, therapeutic or preventive toward treating any medical condition or disease (i.e. cancer, viral diseases, bacterial diseases, protozoal diseases, immune disorders, hormone disorders, neurological diseases and heart diseases) or inhibitory or toxic toward any disease causing organism, especially intracellular organisms that include viruses, bacteria, mycoplasma, protozoa, fungi, parasites and prions. Protein or peptide active agents are further limited to the following categories.

### Chloroquine Combinative Agents

In this invention, preferred protein or peptide active agents are “chloroquine combinative” active agents or a chloroquine combinative agent (CCA) that is preferably coupled with a chloroquine substance defined herein. A CCA is defined as an active agent whose effectiveness or mode of action is potentially amplified or improved or potentially synergistic when used before, during or after treatment with any chloroquine substances, defined herein.

Protein CCA or Peptide CCA.

Protein CCAs or peptide CCAs are defined here as various protein active agents or peptide active agents, pharmaceutical proteins, polypeptides, bioactive peptides, peptide aptamers defined herein whose delivery, effectiveness or mode of action is amplified or improved or synergistic when coupled with any chloroquine substances, defined herein.

Protein or peptide CCAs include cyclosporins, ricins, ricins A, B, C and D including extracts such as RCL I, II, III and IV, saporins including saporin-6 and other ribosome inactivating proteins, tyrocidines and bungarotoxins, among others.

Preferred protein or peptide CCAs include pro-apoptotic peptides including the mitochondrial polypeptide called Smac/Diablo, or a region from the pro-apoptotic proteins called the BH3 domain and other pro-apoptotic peptides.

Preferred protein CCAs or peptide CCAs also include polypeptide hormones, calcitonins, enkephalins, erythropoietin (EPO), EPO derivatives, follical stimulating hormone (FSH), FSH derivatives, human growth hormone (HGH), HGH derivatives, glucagons, gonadotropin-releasing hormones, human insulin and other insulins, insulin fragments, pegylated insulin and other insulin derivatives, interferons (i.e. alpha, beta, or gamma interferons), pegylated interferons, interleukins, pegylated interleukins, laminin fragments, tumor necrosis factors and TNF superfamily of proteins (i.e. TNF, TNF alpha, TNF beta, TNFα, 4-IBBL, APRIL, BAFF, CD27L, CD30L, CD40L, FasL, LIGHT, OX40L, RANKL, TRAIL, TWEAK and VEG1).

Preferred protein or peptide CCAs also include certain vaccine antigens.

Preferred protein or peptide CCAs also include, but are not limited to, any pharmaceutical proteins, polypeptides or bioactive peptides (i.e. interferons, insulins, FSH, HGH, TNF) containing or coupled (i.e. by recombinant methods) to Fc-fusion proteins, peptides or other moieties including those employing the neonatal Fc receptor (FcRn) as disclosed by Syntronix Pharmaceuticals, Waltham Mass., USA, including Synfusion™ and Transceptor™ technologies for protecting and/or transporting proteins across epithelial cell barriers such as in the lungs and intestines.

Preferred protein or peptide CCAs also include, but are not limited to, any pharmaceutical proteins, polypeptide hormones or bioactive peptides defined herein that are “dominant negative” (DN), meaning they have been engineered to eliminate receptor affinity yet retain specific ligand affinity for therapeutic effect. Preferred examples include, but are not limited to DN-TNF disclosed by Xencor, Inc., Monrovia Calif., USA, and any DN engineered forms of the TNF superfamily of proteins (i.e. TNF, TNF alpha, TNF beta, TNFα, 4-1BBL, APRIL, BAFF, CD27L, CD30L, CD40L, FasL, LIGHT, OX40L, RANKL, TRAIL, TWEAK and VEG1).

Antibody CCA. Preferred protein CCAs include any antibody substance, defined herein, including synthetic antibodies, therapeutic antibodies, which includes all types of antibodies disclosed or referenced herein that are useful against any disease or disorder.

Antibody Substance. For this invention, antibody substance is meant to include all antibodies, antibody derivatives and antibody-like substances, such as recombinant and/or chemically engineered substances with antibody origins. For this invention, preferred antibody substances are therapeutic agents and/or targeting moieties that may also function as protein carrier substances for additional active agents and moieties. Preferred antibody substances include antibodies from any animal or biological source, including all classes of antibodies (i.e. IgG, IgE, IgM, including all subclasses, gamma globilins, among others), rhonoclonal antibodies, chimeric antibodies, oxidized antibodies, recombinant antibodies, humanized antibodies, synthetic antibodies, therapeutic antibodies, pegylated antibodies, Fab fractions, antibody fragments, monovalent antibody fragments (Fab, scFv) and engineered variants (i.e. diabodies, triabodies, minibodies and single-domain antibodies), antibody drug conjugates (ADC) that include, but are not limited to, antibodies coupled with Fc-associated N-linked oligosaccharides, protein toxins, radionuclides, and anticancer drugs and derivatives thereof. Antibody substances are distinguishable by their structure and function and are defined here under distinct categories or types.

Most preferred antibody substances include, but are not limited to, any therapeutic antibodies, Alemtuzumab (Campath-1H), BEC2 (mitumomab), hCD22 (epratuzumab), Avastin™ (bevacizumab), Brevarex™, CDP860, Herceptin™ (trastuzumab), HuMax-CD20, HuMax-CD4, HuMax-EGFr, huN901-DM1, IDEC-114, IGN-101, MLN2704 (bivatuzumab mertansine), MLN591RL, Mylotarg™ (gemtuzumab ozogamicin), Omnitarg™ (pertuzumab), Orthoclone OKT3, OvaRex, R1549 (pemtumomab), Raptiva™, Reopro™, Rituxan™ (rituximab), SGN-15, SGN-30, SGN-35, SGN-75, Simulect™, Synagis™, TheraCIM hR3, Tysabri™, Vitaxin™, Xolair™ and Zenapax™, including fractions and derivatives thereof.

Preferred antibody substances also include, but are not limited to, RAV12 (from Raven Biotechnologies, S. San Francisco, Calif. USA, an IgG1 chimeric antibody recognizing an N-linked carbohydrate epitope expressed on human carcinomas); CAT-3888, CAT-8015, CAT-354, GC-1008, HUMIRA® (adalimumab), ABT-874, LymphoStat-B™, HGS-ETR1, HGS-ETR2, ABthrax™, MYO-029, MT201, IMC-11F8 and IMC-1121B (from Cambridge Antibody Technology, Cambridge, Mass.); Ch14.18. chimeric mAb, Rencarex (WX-9250; cG250) chimeric mAb, MDX-010 humanized mAb, Panitumumab (ABX-EGF) human mAb, Remitogen (Hu1D10) humanized mAb and Remicade® (infliximab), including fractions and derivatives of these substances.

Preferred antibody substances also include, but are not limited to, any antibodies disclosed by Acceptys Inc., Sparta, N.J. 07871, including LM-1, NORM-1, NORM-2, SAM-6, CM-1, CM-2, PM-1 and PM-2, among others, and;

any anti bacterial, antiviral and anti fungal antibodies, and antibody conjugates (i.e. anti-RBC receptor (CR1) antibody coupled to an anti-bacterial antibody) such as those disclosed by Elusys Therapeutics Inc., Pine Brook, N.J. 07058, including heteropolymer (HP) antibodies, including Anthim™ and ETI-211, among others, and;

those disclosed by Imclone Systems Inc., New York, N.Y. 10014, including Erbitux™ (cetuximab), Flt-3 mAb, VEGFR-3 mAb, VE-cadherin mAb, FGFR mAb, Ron mAb, VE-cadherin mAb, TRP-1 mAb, PDGFRβ mAb and Neuropilin mAb, among others, and;

those disclosed by Medarex Inc., Princeton N.J., USA, including MDX-010 (ipilimumab), MDX-1379 (anti-CTLA4), HuMax-CD4 (zanolimumab, anti-CD4), CNTO 148 (golimumab, anti-TNFα), CNTO 1275 (anti-IL12/1L23), MDX-066 (CDA-1, anti-C. difficile Toxin A), MDX-060 (anti-CD30), MDX-070 (anti-PSMA), HuMax-CD20 (anti-CD20), AMG 714 (anti-IL15), MDX-214 (anti-EGFR/CD89), MDX-018, (Undisclosed), HuMax-EGFR (anti-EGFR), CNTO 95 (anti-integrin receptors), MDX-1307 (anti-Mannose Receptor/hCGβ), NVS Antibody #1, NVS Antibody #2, FG-3019 (anti-CTGF), HGS-TR2J (anti-TRAIL-R2), LLY Antibody, MDX-1100 (anti-IP10), MDX-1303 (Valortim™, anti-B. anthracis), MEDI-545 (MDX-1103, anti-IFNα), BMS-66513, NI-0401 (anti-CD3), MDX-1333 (anti-IFNAR), MDX-1106 (anti-PD1) and MDX-1388 (anti-C. difficile Toxin B) and;

those disclosed by MedImmune, Inc., Gaithersburg, Md. 20878, including hMPV mAb, siplizumab, Anti-EphA2 mAb, Anti-EphA4 mAb, Anti-EphB4 and EphrinB2 MAbs, Anti-ALK mAb, Anti-IL-9 mAb, Anti-IFNa mAb, Anti-HMGB-1 mAb, Anti-IFNaR mAb, Anti-Chitinase mAb, Anti-CD19, CD20 and CD22 MAbs, among others, and;

those disclosed by PDL BioPharma, Inc., Fremont, Calif. 94555, including Nuvion® (visilizumab), Zenapax® (daclizumab), Volociximab and HuZAF™ (fontolizumab), among others, including fractions and derivatives of these substances.

Preferred antibody substances also include, but are not limited to, any antibodies containing or coupled (i.e. by recombinant methods) to Fc-fusion proteins, peptides or other moieties including those employing the neonatal Fc receptor (FcRn) as disclosed by Syntronix Pharmaceuticals, Waltham Mass., USA, including Synfusion™ and Transceptor™ technologies for protecting antibodies and/or transporting antibodies across epithelial cell barriers such as in the lungs and intestines, including fractions and derivatives thereof.

Immunoconjugates are also preferred antibody substances wherein the antibody substance is conjugated to any suitable drug including auristatins (monomethylauristatin E, monomethylauristatin F, and derivatives) calicheamicins and/or is radiolabled (i.e. with ¹³¹I, ³⁰Y) Preferred examples include, but are not limited to, Gemtuzumab (Myelotarg™), ⁹⁰Y Ibritumomab tiuxetan (Zevalin™) alone or together with rituximab, hAFP-Y-90, hCD22-Y-90 (⁹⁰Y epratuzumab), hCEA-I-131, Tositumomab and ¹³¹I tositumomab (Bexxar™), including fractions and derivatives of these substances.

Preferred antibody substances include, but are not limited to, any antibody substances that bind to any therapeutic targets including but not limited to oncology targets and corresponding antibody substances disclosed by P. Carter, L Smith and M Ryan, Endocrine-Related Cancer (2004) 11 659-687 (cell surface antigens for antibody targeting in oncology, including but not limited to, AFP, aᵥb₃ (vitronectin receptor), CA125 (MUC16), CD4, CD20, CD22 (Siglec-2), CD30 (TNFRSF1), CD33 (Siglec-3), CD52 (CAMPATH-1), CD56 (NCAM), CD66e (CEA), CD70, CD80 (B7-1), CD140b (PDGFRb), CD152 (CTLA4), CD227 (PEM, MUC1, mucin-1), EGF receptors (HER1, ErbB1), HER2 (HER2/neu, ErbB2), EpCam, anti-idiotype vaccine, GD3 ganglioside (anti-idiotype vaccine), PSMA, Sialyl LewisY and VEGF), including all references therein, all the contents of which are incorporated herein.

Preferred antibody substances include, but are not limited to, any antibody substances that are antiviral or antibacterial, such as those that bind to the PcrV protein of the type III secretion system (KaloBios).

Preferred antibody substances include, but are not limited to, any antibody substances that bind to the Fc Receptor on effector cells, wherein the crystallisable fragment (Fc) region elicits the Antigen Dependent Cell-mediated Cytotoxicity (ADCC) response and/or the plasma-native Complement Dependent Cytotoxicity (CDC) response and/or apoptosis.

Preferred antibody substances include, but are not limited to, recombinant antibodies for techniques such as “Antibody-Directed Enzyme Prodrug Therapy” (ADEPT), that conjugate the specificity of antibodies to a prodrug-catalytic subunit (i.e. enzyme) thus creating a high local concentration of an activated chemotherapeutic. Preferred ADEPT antibody substances include, but are not limited to those disclosed by S. K. Sharma, et al, Curr. Opin. Investig. Drugs (2005) 6, 611-615 and N. O. Siemers, et al, Bioconj. Chem. (1997) 8, 510-519; including all references therein, all the contents of which are incorporated herein.

Antibody substances include those used to recruit the adaptive immune response through antibody fragments with a recombinant MHC molecule displaying a highly immunogenic peptide. Preferred antibody substances include, but are not limited to, any antibody substances composed of linked antibody fragments, and altered glycosylation antibodies.

Preferred antibody substances include, but are not limited to, immunotoxins, and antibody conjugates and preparation methods disclosed by K J Hamblet, et al, Clin. Can. Res. (2004) 10, 7063-7070, W Mao, et al, Can. Res. (2004) 64, 781-788, S Doronina, et al, Nat. Biotechnol. (2003) 21: 778-784, 1 Pastan, et al, Curr Opin Investig Drugs (2002) 3(7):1089-91 and RL Shields, et al, J Biol Chem (2002) 277(30) 26733-40, including all references therein, all the contents of which are incorporated herein.

Preferred antibody substances include, but are not limited to, immunotoxins, antibody conjugates, antibody fragments, and their preparation methods and treatment methods disclosed by; Adams G P, Weiner L M., Nat. Biotechnol. (2005) 23(9):1147-57; Wu, A M & P D Senter, Nature Biotechnol. 23, 1137-1146 (2005) (immunoconjugates); Holliger, P & P J Hudson, Nature Biotechnol. 23, 1126-1136 (2005) (engineered antibodies including smaller recombinant antibody fragments, i.e. monovalent antibody fragments (Fab, scFv) and engineered variants (diabodies, triabodies, minibodies and single-domain antibodies)); Monya Baker, Nature Biotechnology 23, 1065-1072 (2005) (linked fragments, altered glycosylation); Schaedel, O and Reiter Y., Curr Pharm Des. (2006) 12(3):363-78 (antibodies and their fragments useful in ADCC, CDC and ADEPT); Giammona, G, et al, Adv Drug Deliv Rev. (1999) 39(1-3):153-164 and Paulik M, et al, Biochem Pharmacol. (1999) 58(11):1781-90 (AZT, anti-transferrin receptor conjugated antibody (OX-26), AZT and alpha, beta-poly(N-hydroxyethyl)-DL-aspartamide (PHEA) conjugates, HIV-specific peptide antibody-brefeldin A conjugates and antibody-glaucarubolone conjugates); including all references therein, all the contents of which are incorporated herein.

Preferred antibody substances include, but are not limited to, immunotoxins, antibody conjugates, antibody fragments, and their preparation methods and treatment methods disclosed by; C. D. Austin, et al, PNAS (2005) 102;17987-17992 (disulfide-based antibody-drug conjugates); S. O. Doronina, et al, Bioconjugate Chem., 17; 114-124 (2006) (antibody drug linkers); M. M. C. Sun, et al, Bioconjugate Chem., 16 (5), 1282-1290, (2005) (reduction-alkylation strategies for antibody conjugates); including all references therein, all the contents of which are incorporated herein.

Preferred antibody substances also include, but are not limited to those that bind to specific cell receptors such as anti-transferrin antibodies used to cross the blood brain barrier. Such BBB-penetrating antibodies are limited to those with affinity to specific transferrin receptors, or other receptors of the BBB such as the lactotransferrin receptor in humans.

Preferred antibody substances also include, but are not limited to, domain antibodies (dAbs), which are the smallest functional binding units of antibodies, corresponding to the variable regions of the heavy (VH) or light (VL) chains, such as those disclosed by Domantis, Cambridge, UK. Preferred domain antibodies include dual targeting dAbs that include: IgG-like molecules that can bind and neutralise up to four (or more) target molecules; PEGylated fusion proteins; and anti-serum albumin fusion proteins. Preferred dAbs include but are not limited to, dAbs with a tailored serum half life, dAbs for pulmonary or oral administration for lung or GI tract diseases and so-called “AlbudAb” substances that include dAb heterodimers that include an anti-serum albumin dAb that confers a long half-life via a serum albumin binding carrier effect.

Synthetic Antibody.

Preferred antibody substances include but are not limited to, synthetic antibodies, defined as antibody derivatives or genetically engineered antibodies including but not limited to antibody fusion proteins including antibody-avidin and antibody-streptavidin fusion proteins including but not limited to those disclosed by M L Penichet, et al, J. Immunol. (1999) 163(8):4421-6; J. Schultz, et al, Cancer Res. (2000) 60, 6663-6669 (tetravalent single chain antibody-streptavidin fusion proteins); S. Goshom, et al, Cancer Biother. Radiopharm.(2001) 16, 109-123 (humanized antibody-streptavidin fusion proteins); E. A. Rossi, et al, Clin. Cancer Res. (2005) (trivalent bispecific antibody fusion proteins); including all references therein, all the contents of which are incorporated herein. Synthetic antibodies include chimeric antibodies, Fab fractions of antibodies, antibody fragments and derivatives thereof and may be included in the category of targeting moieties.

Foldamer CCA.

The foldamer CCAs are defined here as any synthetic pharmaceutical and bioactive oligomers and “protein mimics” including oligomers of beta-peptides, including those disclosed by A. Schepartz, et al, in J. Am. Chem. Soc. 129, p. 1532 (2007) and references therein, that are inhibitory, antimetabolic, therapeutic or preventive toward any disease (i.e. cancer, syphilis, gonorrhea, influenza and heart disease) or inhibitory or toxic toward any disease causing agent.

Antiviral Protein or Peptide CCA.

Preferred antiviral protein or peptide CCAs include, but are not limited to, any enzyme inhibitors including;

S-adenosylhomocysteine (SAH) hydrolase inhibitors, any anti human immunodeficiency virus (HIV) agents, any anti influenza agents, including any protease inhibitors such as inhibitors of FIV and HIV proteases.

Preferred antiviral CCAs include, but are not limited to, any non-nucleoside reverse transcriptase inhibitors (NNRTIs); any entry inhibitors (including fusion inhibitors); any maturation inhibitors; any neuraminidase (NA) inhibitors and any ion channel blockers.

Preferred antiviral protein or peptide CCAs include, but are not limited to, those useful against severe acute respiratory syndrome human coronavirus (SARS) and those useful against human respiratory syncytial virus (HRSV) and include, but are not limited to, any synthetic peptides including those containing amino acids 77 to 95 (especially peptides 80-90) of the intracellular GTPase RhoA including but not limited to, those of P. J. Budge, et al, Antimicrob Agents Chemother. 47(11): 3470-7 (2003); including references therein.

Preferred antiviral protein or peptide CCAs include, but are not limited to, any suitable drugs useful against adenoviruses, adeno-associated viruses (AAV), alphaviruses, arenaviruses, coronaviruses, cytomegalovirus (CMV), flaviviruses, hepatitis viruses, herpesviruses, (oral & genital herpes), herpes zoster virus (shingles), human papiloma virus (HPV, genital warts, anal/cervical cancer), Molluscum Contagiosum, oral hairy leukoplakia (OHL), myxoviruses, oncornaviruses, papovaviruses, paramyxoviruses, parvoviruses, picomaviruses (poliovirus, coxsackievirus, echovirus), poxviruses, reoviruses, rhabdoviruses, rhinoviruses, togaviruses, viroids and any other viral diseases, including drug analogs and derivatives thereof.

Antimicrobial CCA.

Preferred antimicrobial protein or peptide CCAs include, but are not limited to, any suitable antibiotic described or referenced herein including analogs and derivatives thereof. Antimicrobial CCAs include but are not limited to antibacterial, antifungal and antiprotozoan substances including various antibiotics including derivatives and analogs such as antibiotic peptides (i.e. bacitracin, capreomycin, polymyxin B, polymyxin E, tyrothricin, vancomycin).

Preferred antimicrobial protein or peptide CCAs also include, but are not limited to, any suitable drugs useful against acinetobacter, achromobacter, actinomycetes, bacterial diarrhea (Salmonellosis, Campylobacteriosis, Shigellosis), bacterial pneumonia, bacteroides, clostridium chlamydia, corynebacteria, enteric bacilli, gram-negative bacteria, gram-positive bacteria, hemophilus-bordetella bacteria, lactobacillus, mycobacteria, (M. Avium Complex, MAC), Mycobacterium Kansasii, any mycoplasma, neisseria, spirochetes, syphilis, neurosyphilis, pneumococci, rickettsia, staphylococci, streptococci, tuberculosis (TB) and any other bacterial diseases, including analogs and derivatives thereof.

Preferred antimicrobial protein or peptide CCAs also include, but are not limited to, fungicides, antimycotics including any antifungal agents useful against any mycoses, ascomycetes, aspergillus, basidiomycetes, blastomyces, candida, candidiasis (thrush, yeast infection), coccidioidomycosis, coccidiodes, cryptococcus, cryptococcal meningitis, deuteromycetes, histoplasma, paracoccidiodes, phycomycetes, other yeasts and any other fungal diseases, including analogs and derivatives thereof.

Preferred antimicrobial protein or peptide CCAs also include, but are not limited to, antimicrobials and antimalarials including any antiprotozoan agents or drugs useful against any protozoan organisms or their diseases, amebiasis, cryptosporidiosis, isosporiasis, leishmaniasis, malaria, microsporidiosis, pneumocystis pneumonia (PCP), toxoplasmosis, and other protozoan diseases; pesticides, including analogs and derivatives thereof.

Antiparasitic CCA.

Preferred antiparasitic protein or peptide CCAs include, but are not limited to, any suitable drugs useful against any parasites including round worms, flat worms, tape worms, fluke worms, any parasitic arthropods including ticks, insects, mites, and any other parasites, including analogs and derivatives thereof.

Immune Disorder Protein or Peptide CCA.

Preferred protein or peptide CCAs also include those used for prophylaxis or treatment against any immunological or autoimmune diseases including rheumatoid arthritis, systemic lupus erythematosus (SLE), inflammatory bowel disease. (IBD) graft-versus-host diseases, stem cell therapy and diabetes mellitus. Preferred protein or peptide CCAs also include those used for prophylaxis or treatment against any immune-related neurological diseases (i.e. multiple sclerosis, Alzheimer's, Parkinson's), heart diseases, prion diseases and cancers.

Immune disorder protein or peptide CCAs include but are not limited to, any anti-inflammatory protein or peptide drugs. Preferred immune disorder CCAs include but are not limited to, any agents used to treat rheumatoid arthritis including any “disease modifying antirheumatic drug” (DMARD.

Neurological Protein or Peptide CCA.

Preferred protein or peptide CCAs include certain neurological proteins and peptides, any antidepressant drugs, any analgesic, anesthetic and neurologic drugs.

Anticancer Protein or Peptide CCA.

Preferred CCAs are “anticancer combinative agents” defined as any antineoplastic protein or peptide agents, prodrugs or cell growth inhibitors that are potentially enhanced when combined with chloroquine substances. Preferred anticancer CCAs include but are not limited to, agents against drug resistant forms of cancer that rely on inhibition of apoptosis or on endosomal mechanisms to excrete active agents. These also include, but are not limited to; aromatase inhibitors; EGFR tyrosine kinase inhibitors and aurora kinase inhibitors.

### Other Terms and Definitions

Pharmaceutical.

For the purposes of this invention, pharmaceutical or “pharmaceutical use” is defined as being limited to substances that are useful or potentially useful in therapeutic or prophylactic applications against diseases or disorders in humans, or any other vertebrate animals and in plants, especially plants of economic value. The most preferred substances defined as pharmaceutical are substances and/or compositions useful against viral, bacterial, fungal, protozoan, parasitic and other disease organisms, against cancers, autoimmune diseases, genetic diseases, heart diseases, neurological diseases and other diseases or disorders in humans and other vertebrates. Generally, but not necessarily, pharmaceutical substances are also biocompatible.

Biocompatible is defined here to mean substances that are suitably designed to be generally non-immunogenic, non-antigenic and will cause minimum undesired physiological reactions. They may or may not be degraded biologically and they are suitably “biologically neutral” for pharmaceutical applications due to suitably low, non-specific binding properties.

Coupling.

For the instant invention, two distinct types of coupling are defined to produce different compositions. One type of coupling can be through noncovalent, “attractive” binding as with a guest molecule and cyclodextrin, an intercalator and nucleic acid, an antigen and antibody, biotin and avidin, or noncovalent coupling can be between an antibody and a micelle or nanoparticle containing other moieties either covalently or noncovalently coupled. Such noncovalent coupling is binding between substances through ionic or hydrogen bonding or van der waals forces, and/or their hydrophobic or hydrophilic properties.

Unless stated otherwise, the preferred coupling used in the instant invention is through covalent, electron-pair bonds or linkages. Many methods and agents for covalently coupling (or cross linking) of carrier substances including polyethylene glycol and other polymers are known and, with appropriate modification, can be used to couple the desired substances through their “functional groups” for use in this invention. Where stability is desired, the preferred covalent linkages are amide bonds, peptide bonds, ether bonds, and thio ether bonds, among others.

Functional Group.

A functional group or reactive group is defined here as a potentially reactive moiety or “coupling site” on a substance where one or more atoms are available for covalent coupling to some other substance. When needed, functional groups are added to a carrier substance such as polyethylene glycol through derivatization or substitution reactions.

Examples of functional groups are aldehydes, allyls, amines, amides, azides, carboxyls, carbonyls, epoxys (oxiranes), ethynyls, hydroxyls, phenolic hydroxyls, indoles, ketones, certain metals, nitrenes, phosphates, propargyls, sulfhydryls, sulfonyls, vinyls, bromines, chlorines, iodines, and others. The prior art has shown that most, if not all of these functional groups can be incorporated into or added to the carrier substances of this invention.

Pendant Functional Group.

A pendant or “branched” functional or reactive group is defined here as a functional group or potentially reactive moiety described herein, that is located on a suitable polymer backbone such as pendant polyethylene glycol and “comb shaped” polymers, between the two ends. Preferably the pendant functional groups are located more centrally than peripherally.

Linkage.

A linkage is defined as a chemical moiety within the compositions disclosed that results from covalent coupling or bonding of the substances disclosed to each other. A linkage may be either biodegradable or non-biodegradable and may contain suitable “spacers' defined herein. Suitable linkages are more specifically defined below.

Coupling Agent.

A coupling agent (or cross-linking agent), is defined as a chemical substance that reacts with functional groups on substances to produce a covalent coupling, or linkage, or conjugation with said substances. Because of the stability of covalent coupling, this is the preferred method. Depending on the chemical makeup or functional group on a carrier substance, amphiphilic molecule, cyclodextrin, or targeting molecule, the appropriate coupling agent is used to provide the necessary active functional group or to react with the functional group. In certain preparations of the instant invention, coupling agents are needed that also provide a linkage with a “spacer” or “spacer arm” as described by O'Carra, P., et al, FEBS Lett. 43, 169 (1974) between a carrier substance and an intercalator or targeting molecule to overcome steric hindrance. Preferably, the spacer is a substance of 4 or more carbon atoms in length and can include aliphatic, aromatic and heterocyclic structures.

With appropriate modifications by one skilled in the art, the coupling methods referenced in U.S. Pat. No. 6,048,736 and PCT/US99/30820, including references contained therein, are applicable to the synthesis of the preparations and components of the instant invention and are hereby incorporated by reference.

Examples of energy activated coupling agents are ultraviolet (UV), visible and radioactive radiation that can promote coupling or cross linking of suitably derivatized substances. Examples are photochemical coupling agents disclosed in U.S. Pat. No. 4,737,454, among others. Useful derivatizing and/or coupling agents for preparing polymers are bifunctional, trifunctional or polyfunctional cross linking agents that will covalently couple to the functional groups of suitable monomers and other substances.

Useful in this invention are coupling agents selected from the group of oxiranes or epoxides. Some preferred examples of oxiranes and epoxides include; epichlorohydrin, 1,4 butanediol diglycidyl ether (BDDE), bis(2,3-epoxycyclopentyl) ether 2,2′-oxybis(6-oxabicyclo[3.1.0] hexane) (BECPE), glycerol diglycidyl ether (GDE), trimethylolpropane triglycidyl ether (TMTE), tris(2,3-epoxypropyl) isocyanurate (TEPIC), glycerol propoxylate triglycidyl ether (GPTE), 1,3-butadiene diepoxide, triphenylolmethane triglycidyl ether, 4,4′-methylenebis (N,N-diglycidylaniline), tetraphenylolethane glycidyl ether, bisphenol A diglycidyl ether, bisphenol A propoxylate diglycidyl ether, bisphenol F diglycidyl ether, cyclohexanedimethanol diglycidyl ether, 2,2′-oxybis (6-oxabicyclo[3.1.0] hexane), polyoxyethylene bis(glycidyl ether), resorcinol diglycidyl ether, ethylene glycol diglycidyl ether (EGDE) and low molecular weight forms of poly(ethylene glycol) diglycidyl ethers or poly(propylene glycol) diglycidyl ethers, among others.

Other preferred derivatizing and/or coupling agents for hydroxyl groups are various disulfonyl compounds such as benzene-1,3-disulfonyl chloride and 4,4′-biphenyl disulfonyl chloride and divinyl sulfone (J. Porath, et al, J. Chromatog. 103, 49-62, 1975), among others.

Most preferred coupling agents are also chemical substances that can provide the bio-compatible linkages for synthesizing the compositions of the instant invention. Covalent coupling or conjugation is done through functional groups using coupling agents such as glutaraldehyde, formaldehyde, cyanogen bromide, azides, p-benzoquinone, maleic or succinic anhydrides, carbodiimides, ethyl chloroformate, dipyridyl disulfide and polyaldehydes.

Also most preferred are derivatizing and/or coupling agents that couple to thiol groups (“thiol-reactive”) such as agents with any maleimide, vinylsulfonyl, bromoacetal or iodoacetal groups, including any bifunctional or polyfunctional forms. Examples are m-maleimidobenzoyl-N-hydroxysuccinimide ester (MBS), succinimidyl 4-(N-maleimidomethyl) cyclohexane-1-carboxylate (SMCC), succinimidyl 4-(p-maleimidophenyl)butyrate (SMPB), dithiobis-N-ethylmaleimide (DTEM), 1,1′-(methylenedi-4,1-phenylene) bismaleimide (MPBM), o-phenylenebismaleimide, N-succinimidyl iodoacetate (SIA), N-succinimidyl-(4-vinylsulfonyl) benzoate (SVSB), and tris-(2-maleimidoethyl) amine (TMEA), among others.

Other coupling groups or agents useful in the instant invention are: p-nitrophenyl ester (ONp), bifunctional imidoesters such as dimethyl adipimidate (DMA), dimethyl pimelimidate (DMP), dimethyl suberimidate (DMS), methyl 4-mercaptobutyrimidate, dimethyl 3,3′-dithiobis-propionimidate (DTBP), and 2-iminothiolane (Traut's reagent);

bifunctional tetrafluorophenyl esters (TFP) and bifunctional NHS esters such as disuccinimidyl suberate (DSS), bis[2-(succinimido-oxycarbonyloxy) ethyl]sulfone (BSOCOES), disuccinimidyl (N,N′-diacetylhomocystein) (DSAH), disuccinimidyl tartrate (DST), dithiobis(succinimidyl propionate) (DSP), and ethylene glycol bis(succinimidyl succinate) (EGS), including various derivatives such as their sulfo-forms;

heterobifunctional reagents such as p-nitrophenyl 2-diazo-3,3,3-trifluoropropionate, N-succinimidyl-6(4′-azido-2′-nitrophenylamino) hexanoate (Lomant's reagent II), and N-succinimidyl-3-(2-pyridyldithio)propionate (SPDP), including various derivatives such as their sulfo-forms;

homobifunctional reagents such as 1,5-difluoro-2,4-dinitrobenzene, 4,4′-difluoro-3,3′-dinitrophenylsulfone, 4,4′-diisothiocyano-2,2′-disulfonic acid stilbene (DIDS), p-phenylene-diisothiocyanate (DITC), carbonylbis(L-methionine p-nitrophenyl ester), 4,4′-dithiobisphenylazide and erythritolbiscarbonate, including derivatives such as their sulfo-forms;

photoactive coupling agents such as N-5-azido-2-nitrobenzoylsuccinimide (ANB-NOS), p-azidophenacyl bromide (APB), p-azidophenyl glyoxal (APG), N-(4-azidophenylthio) phthalimide (APTP), 4,4′-dithio-bis-phenylazide (DTBPA), ethyl 4-azidophenyl-1,4-dithiobutyrimidate (EADB), 4-fluoro-3-nitrophenyl azide (FNPA), N-hydroxysuccinimidyl-4-azidobenzoate (HSAB), N-hydroxysuccinimidyl-4-azidosalicylic acid (NHS-ASA), methyl-4-azidobenzoimidate (MABI), p-nitrophenyl-2-diazo-3,3,3-trifluoropropionate (PNP-DTP), 2-diazo-3,3,3-trifluoropropionyl chloride, N-succinimidyl-6(4′-azido-2′-nitrophenylamino) hexanoate (SANPAH); N-succinimidyl(4-azidophenyl)1,3′-dithiopropionate (SADP), sulfosuccinimidyl-2-(m-azido-o-nitobenzamido)-ethyl-1,3′-dithiopropionate (SAND), sulfosuccinimidyl (4-azidophenyldithio) propionate (Sulfo-SADP), sulfosuccinimidyl-6-(4′-azido-2′-nitrophenylamino) hexanoate (Sulfo-SANPAH), sulfosuccinimidyl-2-(p-azido salicylamido) ethyl-1,3′-dithiopropionate (SASD), and derivatives and analogs of these reagents, among others. The structures and references for use are given for many of these reagents in, “Pierce Handbook and General Catalog”, Pierce Chemical Co., Rockford, Ill., 61105.

Biocleavable Linkage or Bond.

For the instant invention, biocleavable linkages are defined as types of specific chemical moieties or groups that can be used within the compositions to covalently couple or cross-link a carrier substance or chloroquine substance with the antibody substances, protein or peptide active agents, targeting moieties, amphiphilic molecules and grafted polymers described herein. They may also be used in certain embodiments of the instant invention to provide the function of controlled release of chloroquines and/or protein or peptide active agents. Some suitable, but not limited to, examples of linkages useful in this invention (including use in oral delivery) are disclosed by V. R. Sinha, et al, Europ. J Pharmaceut. Sci. 18, 3-18 (2003), including references therein. Also useful, but not limited to, in this invention are linkages (and their synthesis methods) used for coupling prodrugs to polymeric carriers for subsequent release, including those disclosed by A. Joseph, et al, J. Pharm. Sci. 93, 1962-1979 (2004) and K. Hoste, et al, Int. J. Pharmacol. 277, 119-131 (2004), including references therein and are incorporated herein. Biocleavable linkages or bonds are distinguishable by their structure and function and are defined here under distinct categories or types.

Ester Linkages. The ester bond is a preferred type that includes those between any carboxylic acid and alcohol or hydroxyl group and may be protected by electron-donating effect. Preferred ester bonds include any of the ester bonds used in the preparation of prodrugs or prodrug conjugates including but not limited to disclosures by S. Gunaseelan, et al, Bioconj. Chem. (2004) 15, 1322-1333 and A. Joseph (supra) and V R Sinha, (disclosed herein), including references therein. Examples include carboxylic esters, carbonate esters, carbamate esters, aromatic amides and cis-aconityl amides. Another preferred type is certain imidoesters formed from alkyl imidates. Also included are certain maleimide bonds as with sulfhydryls or amines used to incorporate a biocleavable linkage.

Acid Labile Linkages. Another category in this invention comprises biocleavable linkages that are more specifically cleaved after entering the cell (intracellular cleavage). The preferred biocleavable linkages for release of active agents and other moieties within the cell are cleavable in acidic conditions like those found in lysosomes. One type is an acid-sensitive (or acid-labile) hydrazone linkage as described by Greenfield, et al, Cancer Res. 50, 6600-6607 (1990), and references therein and C═N linkages (hydrazones), disclosed by A. Joseph, et al (supra), and references therein. Another type of preferred acid-labile linkage is any type of ortho ester, polyortho or diortho ester linkage, examples disclosed by J. Heller, et al., Methods in Enzymology 112, 422-436 (1985), J. Heller, J. Adv. Polymer Sci. 107, 41 (1993), M. Ahmad, et al., J. Amer. Chem. Soc. 101, 2669 (1979) and references therein. Also preferred are acid labile phosphonamide linkages disclosed by J. Rahil, et al, J. Am. Chem. Soc. 103, 1723 (1981) and J. H. Jeong, et al, Bioconj. Chem. 14, 473 (2003). Another preferred category is certain aldehyde bonds subject to hydrolysis that include various aldehyde-amino bonds (Schiff s base), or aldehyde-sulfhydryl bonds.

Cleavable Peptide Linkages. Another preferred category of biocleavable linkages is biocleavable peptides or polypeptides from 2 to 100 residues in length, preferably from 2 to 20 amino acid residues in length (and can include citrulline (Cit)). These are defined as certain natural or synthetic polypeptides that contain certain amino acid sequences (i.e. D or L, usually hydrophobic) that are cleaved by proteolytic enzymes such as cathepsins, found primarily inside the cell (intracellular enzymes). Using the convention of starting with the amino or “N” terminus on the left and the carboxyl or “C” terminus on the right, some examples are: any peptides that contain the paired amino acids Phe-Leu, Leu-Phe or Phe-Phe, such as Gly-Phe-Leu-Gly (GFLG); any peptides that contain the amino acid phenylalanine (Phe) in combination with one, two or three other amino acids (i.e. Phe-Ala, Phe-Cit, Phe-Ileu, Phe-Lys, Phe-Val, Phe-Pro, Phe-Met, etc.) and any peptides that contain the amino acid valine (Val) in combination with one, two or three other amino acids (i.e. Val-Ala, Val-Cit, Val-Gly, Val-Ileu, Val-Leu, Val-Val, Val-Phe, Val-Pro, Val-Met, etc.) and other combinations. Preferred examples (among others) include leucine enkephalin derivatives and any cathepsin cleavable peptide linkage sequences disclosed by J. J. Peterson, et al, in Bioconj. Chem., Vol. 10, 553-557, (1999), and references therein and in U.S. patent application Ser. No. 10/923,112 that are incorporated herein by reference.

Preferred peptide linkages or bonds also include any peptide linkages or bonds used in the preparation of prodrugs or prodrug conjugates.

Disulfide Linkage. A preferred category comprises the disulfide linkages that are well known for covalent coupling. For drug delivery they may be more useful for shorter periods in vivo since they are cleaved in the bloodstream relatively easily. A preferred type of biocleavable linkage is any disulfide linkages such as those produced by thiol-disulfide interchange (J. Carlsson, et al, Eur. J. Biochem. 59, 567-572, 1975). Preferred disulfide bonds include any of the disulfide bonds used in the preparation of prodrugs or prodrug conjugates.

Protected Disulfide Linkages. Another preferred type of biocleavable linkage is any “hindered” or “protected” or sterically hindered disulfide bond that inhibits attack from thiolate ions or other cleavage mechanisms. Examples of (but not limited to) such protected disulfide bonds are found in the coupling agents: S-4-succinimidyl-oxycarbonyl-α-methyl benzyl thiosulfate (SMBT) and 4-succinimidyloxycarbonyl-α-methyl-α-(2-pyridyldithio) toluene (SMPT). Another useful hindered disulfide linkage is disclosed by P. E. Thorpe, et al, Cancer Res. 48, 6396-6403 (1988) and the coupling agent SPDB disclosed by Worrell, et al., Anticancer Drug Design 1:179-188 (1986). Also included are certain aryidithio thioimidates, substituted with a methyl or phenyl group adjacent to the disulfide, which include ethyl S-acetyl 3-mercaptobutyrothioimidate (M-AMPT) and 3-(4-carboxyamido phenyldithio) proprio-thioimidate (CDPT), disclosed by S. Arpicco, et al., Bioconj. Chem. 8 (3):327-337 (1997) the foregoing references and references therein are hereby incorporated into this invention.

Azo Linkages. Another preferred type of biocleavable linkage in this invention are any suitable azo linkages and aromatic azo linkages that are cleavable by specific azo reductase activities in the colon as disclosed by J. Kopecek, et al., In: Oral Colon Specific Drug Delivery; D. R. Friend, Ed., pp 189-211 (1992), CRC Press, Boca Raton, Fla. and V R Sinha, (disclosed herein), and references therein.

Gastrointestinal Tract Specific Linkages.

Gastrointestinal tract (GIT) specific linkages are defined for this invention as chemical linkages or bioconjugates between any antibody substances, active agents, prodrugs, nucleic acids, carrier substances and any suitable moiety wherein said linkage is cleavable by bacterial action including bacterial hydrolysis. Preferred examples of GIT specific linkages are disclosed, but not limited to, V R Sinha, et al, Europ. J Pharma. Sci., 18, 3-18 (2003), the contents of which, including references therein, are incorporated herein by reference. Sinha, et al examples include, but are not limited to, azo, aromatic azo, amide, glycosidic, glucuronide, and ester linkages.

Controlled Release.

For this invention, controlled release (or “active release”) is defined as the release of chloroquine substances and/or an antibody from each other, an antibody substance or from a carrier composition. Release of the antibody is by cleavage of certain biocleavable covalent linkages described herein that are used to couple the chloroquines, antibodies or antibody to each other, or to the carrier substance.

### Carrier Substance

The present invention is a composition comprised of a chloroquine substance coupled to protein or peptide active agents, including an antibody substance defined herein, directly or through said carrier substance. Preferably the carrier substance provides or contributes to a biocompatible framework or “backbone” to which are coupled various moieties. For the purposes of this invention, a carrier substance is defined as a molecular moiety suitable for pharmaceutical or diagnostic use that is one of the materials used to synthesize the new carrier compositions of this invention.

This does not include antioxidants, adjuvants or so called pharmaceutical “carriers” or “drug vehicles” defined as pharmaceutical mixtures of solvents, dispersing agents, surfactants, excipients, or their combinations, that comprise a usually aqueous formulation for containing a drug or agent. However, a carrier composition of this invention may include a chemically modified form of a specific substance that has been used in such pharmaceutical mixtures. Also, a carrier composition of this invention may be a useful additive to pharmaceutical mixes.

The carrier substances of this invention are limited by category to a variety of suitable substances including proteins, carbohydrates, grafted polymers and surfactants disclosed herein. The carrier substance can also include combinations of these suitable substances.

### Protein Carrier Substances

Plasma and Cellular Proteins.

Preferred plasma protein carrier substances include any suitable albumins such as human serum albumins (HSA), albumin derivatives (i.e. fractionated, pegylated, methylated, etc.), any HSA derivatized with cis-aconitic anhydride (Aco-HAS) such as disclosed by J A Kamps, et al, Biochim Biophys Acta. (1996) 1278(2):183-90, including references therein, any synthetic albumins and albumins and HSA derived from recombinant protein methods.

Preferred plasma protein carrier substances include serum or plasma proteins including fibrinogens, globulins (gamma globulins, thyroglobulins), haptoglobins and intrinsic factor including their derivatives such as their pegylated forms.

Preferred cellular protein carrier substances include cellular receptors, peptide hormones, enzymes, (especially cell surface enzymes such as neuraminidases) and their derivatives such as their pegylated forms. Preferred cellular protein carrier substances include any suitable histones (such as histones I, II, III and IV, including fragments, sulfates and other derivatives thereof) and histones disclosed by C. Peterson, et al, IN; Current Biology, 14(14); R546-R551 (2004), including references therein.

Protamines.

Preferred cellular protein carrier substances include any suitable protamines including human, fish (such as salmines and clupeines), bovine or other animal or plant protamines including fragments, sulfates and other derivatives thereof (i.e. fractionated, pegylated, methylated, etc.), any synthetic protamines and protamines derived from recombinant protein methods. Also preferred are low molecular weight protamines including any from enzymatic digestion as disclosed by Y. Byun, et al, IN; Thromb. Res. 94; 53-61 (1999), protamine-like proteins disclosed by J. D. Lewis, et al, IN; Biochem. Cell Biol., 80(3); 353-61 (2002), protamines disclosed by J. D. Lewis, et al, IN; Chromosoma, 111(8); 473-82 (2003) and by K. W. Park IN; Int. Anesthesiol. Clin., 42(3); 13545 (2004), including references therein. Also preferred is any suitable protamine that is suitably derivatized to provide a carboxylated carrier substance by reacting it with acetic (or succinic) anhydride in anhydrous solvent.

Noncovalent Coupling Protein

Preferred protein carrier substances include noncovalent coupling proteins which include avidins, streptavidins, staphylococcal protein A, protein G and their fragments, recombinant forms and derivatives including pegylated forms. Avidins and streptavidins are preferred for noncovalent coupling to any suitable biotinylated substance including active agents and chloroquine substances through avidin-biotin linkage.

Antibody Carriers. For this invention, preferred protein carrier substances include any suitable antibody substance, defined herein, and is meant to include all antibodies, gamma globulins, antibody derivatives and antibody-like substances, such as recombinant and/or chemically engineered substances with antibody origins. For this invention, preferred antibody carriers can include therapeutic antibodies and/or targeting antibodies that may also function as protein carrier substances for additional active agents and moieties.

Oxidized Glycoproteins. A preferred category of carrier substances includes glycoproteins that have been suitably oxidized to provide aldehyde functional groups. These include oxidized forms of certain gamma globulins, alpha globulins, mucins, glycopeptides, ovomucoids and other mucoproteins.

Oxidized Antibodies. Another preferred protein carrier substance includes any oxidized forms of antibodies and antibody substances, defined herein, including all classes of antibodies, monoclonal antibodies, chimeric antibodies, pegylated antibodies, fragments and derivatives thereof.

### Peptide Carrier Substances

Preferred carrier substances include any suitable di-, tri-, and poly-peptides including dilysines, trilysines, transduction vectors and receptor binding peptides defined herein. In certain preferred examples, the chloroquine substances and/or intercalators of this invention are coupled to the amphipathic peptide KALA as disclosed by T. B. Wyman, et al, in Biochem. 36, 3008-3017 (1997), which may include derivatives and additional moieties as disclosed herein.

### Carbohydrate Carrier Substances

Preferred carbohydrate carrier substances are carbohydrates including polysaccharides, muco-polysaccharides, and mucoadhesive substances that include alginates, amyloses, dextrans, dextran sulfates, dextrins (alpha-1,4 polyglucose), carrageenans, chitosans, chitosan derivatives, chondroitins, chondroitin derivatives, cyclodextrins, cyclodextrin dimers, trimers and polymers including linear cyclodextrin polymers, gums (i.e. guar or gellan), hyaluronic acids, lectins, hemagglutinins, pectins, inulins and inulin derivatives, any suitable cell wall carbohydrates including zymosans and zymosan derivatives, trisaccharides including raffinose and any pegylated or sulfated carbohydrates or any pegylated or sulfated polysaccharides.

Preferred carrier substances are chitins and chitin derivatives including chitin acetates, chitin sulfates and deacylated chitin such as chitosans including mucoadhesive chitosans for oral delivery. Examples of suitable chitosan carrier substances include, but are not limited to, disclosures by A B Boer, et al, Pharm. Res. 13, 1668-1672 (1996); H Q Mao et al, J Controlled Rel. 70(3), 399421 (2001); A Vila, et al, J Controlled Rel. 78: 15-24 (2002) and J Chen, et al, World J Gastroenterol 10(1): 112-116 (2004), among others. References listed herein, and references therein, are incorporated into this invention by reference.

Preferred examples of carbohydrate carrier substances are polysaccharides disclosed by, but not limited to, V R Sinha, (disclosed herein), dextrin carriers of D. Hreczuk-Hirst, et al, Int J. Pharm. (2001) 230(1-2):57-66, neamine (with or without coupled nucleic acid) of E Riguet, et al, J Med Chem. (2004) 47(20):4806-9 the contents of which, including references therein, are incorporated into this invention by reference. Examples include, but are not limited to polysaccharides or carbohydrates containing, azo, aromatic azo, amide, glycosidic, glucuronide, ester and ortho ester linkages.

### Grafted Polymers

A grafted polymer is a category of carrier substances defined as a polymeric substance suitable for pharmaceutical, diagnostic or agricultural use including copolymers and block polymers such as diblock or triblock copolymers prepared from a variety of monomers that are suitably coupled to produce a carrier substance as defined in the present invention.

Grafted polymers and copolymers can introduce other desirable properties such as a positive or negative net charge and hydrophobic properties. Preferred grafted polymers include cationic grafted polymers, cationic polymers, amphiphilic grafted polymers, amphiphilic molecules and polymers disclosed herein. Preferred grafted polymers are biocompatible, generally hydrophilic and have a molecular weight range from 1000 to 500,000 Daltons, preferably from 2,000 to 200,000 Daltons.

With suitable modification of the synthesis methods referenced by G. S. Kwon, IN: Critical Reviews in Therapeutic Drug Carrier Systems, 15(5):481-512 (1998) and by A. El-Aneed in J. Controlled Rel. 94, 1-14 (2004), including references therein, which are included herein, suitable grafted polymers are synthesized for preparing the compositions of this invention. Included are diblock and triblock copolymer synthesis methods include ring-opening polymerization such as with PEO and various N-carboxyanhydride (NCA) monomers; polymerizations using triphosgenes and organo-metal (i.e. nickel) initiators (i.e. stannous octoate). Also useful are anionic, zwitterionic and free radical polymerizations and transesterifications, among others.

Some examples of suitable substances for use in grafted polymers are certain proteins (such as protamines and histones described herein), polypeptides, polyamino acids, glycoproteins, lipoproteins (i.e. low density lipoprotein), amino sugars, glucosamines, polysaccharides, lipopolysaccharides, amino polysaccharides, polyglutamic acids, poly lactic acids (PLA), polyacrylamides, poly(allylamines), lipids, glycolipids and suitable synthetic polymers, especially biopolymers as well as suitable derivatives of these substances. Also included as suitable substances are the polymers disclosed in U.S. Pat. No. 4,645,646. Also included, but not limited to, in this invention are polymeric carriers (and their synthesis methods) used for coupling to prodrugs for subsequent release, including those disclosed by A. Joseph, et al, J. Pharm. Sci. 93, 1962-1979 (2004) and K. Hoste, et al, Int. J. Pharmacol. 277, 119-131 (2004), including references therein and are incorporated herein.

Preferred grafted polymers include any polyethylene glycols (PEG), PEG derivatives, methoxy polyethylene glycols (mPEG), PEG-polyester carbonates, poly(ethylene-co-vinyl acetate) (EVAc), N-(2-hydroxypropyl) methacrylamides (HPMA), HPMA derivatives, poly(2-(dimethyl amino) ethyl methacrylate (DMAEMA), poly(D, L-lactide-co-glycolide) (PLGA), poly(polypropyl acrylic acid) (PPM), poly (D,L-lactic-coglycolic acid) (PLGA), PLGA derivatives and poly (D,L-lactide)-block-methoxypolyethylene glycol (diblock), polyglutamates (PGA) and any combinations, ratios or derivatives of these.

Preferred grafted polymers in this invention include any polyaspartamides (beta-poly(N-2-hydroxyethyl)-DL-aspartamide, PHEA) and poly-(gamma-D-glutamic) acids (gamma-PGA) that include, but are not limited to, those disclosed by EJF Prodhomme in; Bioconjugate Chem., Vol. 14, No. 6, (2003) and derivatives and references therein.

Also preferred grafted polymers are any copolymers that contain poly(ethylene oxide) (PEO) such as PEO-block-poly(L lysine), PEO-block-poly(aspartate), poly(ethylene glycol)-poly(ester-carbonate) block copolymers, PEO-block-poly(beta-benzyl aspartate), PEO-block-poly(lactic acid), PEO-block-poly(L-lactic-coglycolic acid), poly(propylene oxide) (PPO), PEO-block-PPO and any combinations, ratios and their derivatives.

Preferred grafted polymers include any polyacetals including amino-PEG (APEG), disclosed by R. Tomlinson, et al, Macromolec. 35, 473-480 (2002), amino-pendent polyacetals (APEGs) by R. Tomlinson, et al, Bioconj. Chem. 14, 1096-1106 (2003) and references therein. Preferred grafted polymers include any polymer carriers disclosed by R. Duncan, et al, in Endocrine-Related Cancer (2005) 12, S189-S199 and L Andersson, et al, Biomacromolecules. (2005) 6(2):914-26 including references therein.

Also preferred grafted polymers are any CD dimers, CD trimers, CD polymers and CD blocks, defined herein, poly cyanoacrylates such as poly(butyl cyanoacrylate), poly(isobutyl or isohexyl cyanoacrylate) and any combinations or derivatives of these. Also preferred grafted polymers are comb shaped polymers including N-Ac-poly(L-histidine)-graft-poly(L-lysine) disclosed by J. M. Benns, et al, Bioconj. Chem. 11, 637-645 (2000), and references therein.

Preferred examples of grafted polymer carrier substances are carriers and polymers disclosed by, but not limited to, V R Sinha, (disclosed herein), the contents of which, including references therein, are incorporated into this invention by reference. Grafted polymer examples include, but are not limited to polymers containing, azo, aromatic azo, amide, glycosidic, glucuronide, ester and ortho ester linkages. Preferably, grafted polymers also include any suitable combination of the polymers defined herein.

Amphiphilic Grafted Polymers. Amphiphilic grafted polymers are a preferred category of carrier substances that contain amphiphilic molecules. Amphiphilic molecules are defined as moieties suitable for pharmaceutical or diagnostic use that contain at least one hydrophilic (polar) moiety and at least one hydrophobic (nonpolar) moiety (i.e. surfactant). In certain embodiments of this invention, amphiphilic molecules including amphiphilic block polymers or copolymers are used as the carrier substance or as grafted polymers on the carrier substance.

In one embodiment, the desired chloroquine substance is coupled to one or more available sites on the hydrophilic moieties of an amphiphilic molecule. Then, the chloroquine coupled amphiphilic molecule is incorporated or “anchored” into a micelle containing an antibody substance. The chloroquine substance and antibody substance are thereby noncovalently coupled through the micelle composition of the instant invention.

Most preferred are amphiphilic diblock or triblock copolymers prepared from a variety of monomers to provide at least one hydrophilic and one hydrophobic moiety. Amphiphilic cyclodextrin dimers, trimers and polymers as well as amphiphilic block copolymers containing CD dimers, trimers and polymers are included.

Preferred amphiphilic grafted polymers include any micelle-forming polymers or copolymers including PEG, PEG derivatives, PLGA, PLGA derivatives and poly (D,L-lactide)-block-methoxypolyethylene glycol (diblock), PEO, PEO derivatives or copolymers, PPO and PPO derivatives. Also preferred are any micelle-forming triblock copolymers (Pluronics) that contain PEG, PEO or PPO, such as PEO-block-PPO-block-PEO in various ratios. Specific examples are poloxamer compounds (i.e. TranzFect technology of CytRx Corp., USA); the F, L or P series of Pluronics including, but not limited to, F-68, F-108, F-127, L-61, L-121, P-85, P-105, P-123 and any derivatives.

Cationic Grafted Polymers. Cationic grafted polymers are a preferred category of carrier substances defined as moieties suitable for pharmaceutical or diagnostic use that contain a net positive charge. In certain embodiments of this invention, cationic grafted polymers including cationic block polymers or copolymers are used as the carrier substance or as grafted polymers on the carrier substance. Preferred cationic grafted polymers include, but are not limited to, hexadimethrine bromide (polybrene), polyethylenimine (PEI), polyamidoamines (PAMAM), poly-L-lysine (PLL), poly-L-histidines (PLH), poly ornithines and poly arginines, among others.

### Surfactant Carrier Substances

Preferred surfactant carrier substances include suitable fatty acid derivatives, cholesterol derivatives including cholesterol hemisuccinate morpholine salts (CHEMS), gangliosides, phospholipids, pegylated phospholipids, dioleoylphosphatidyl choline (DOPC), dioleoylphosphatidyl ethanolamine (DOPE), any cationic lipids including 1,2-dioleoyl-3-trimethyl ammonium propane (DOTAP), 1,2-dioleyloxypropyl-3-trimethyl ammonium chloride (DOTMA), 1,2-dimyristyloxypropyl-3-dimethyl-hydroxyethyl ammonium bromide (DMRIE) 1,2-Dioleoyl-3-phosphatidylethanolamine (DOPE), 3 beta-[N-[(N′,N′-dimethylamino)ethane]arbamoyl]cholesterol (DCchol) and other suitable surfactants.

### Liposome

A liposome or vesicle is defined as a water soluble or colloidal structure composed of amphiphilic molecules that have formed generally spherical bilayer membranes. Said amphiphilic molecules are generally oriented in said bilayer membrane so that their hydrophilic ends are on the outside of the membrane and their hydrophobic ends are sequestered inside the membrane. Preferred liposomes contain more than one active agent and are stabilized by crosslinking. They generally have a spherical shape where said bilayer membranes are arranged in one (unilamellar) or more layers (multilamellar) around a single, primarily hydrophilic or aqueous, central zone. Any surrounding membranes may have hydrophilic zones between said membranes around the central hydrophilic zone.

### Micelles and Nanoparticles

A preferred micelle or nanoparticle for this invention is defined as a water soluble or colloidal structure or aggregate (also called a nanosphere) composed of one or more amphiphilic molecules and may include grafted polymers defined herein. Preferred micelles and nanoparticles of this invention generally have a single, central and primarily hydrophobic zone or “core” surrounded by a hydrophilic layer or “shell”. Preferred micelles and nanoparticles of this invention may also be due to aggregation and/or condensation due to self attraction or opposite charge as between an anionic and a cationic substance.

Also preferred are nanoparticles composed of macromolecules including “cascade polymers” such as dendrimers. Preferred dendrimers include polyamidoamines as disclosed by J. Haensler, et al, in Bioconj. Chem. 4, 372-379 (1993) and references therein. Micelles and nanoparticles range in size from 5 to about 2000 nanometers, preferably from 10 to 400 nm. Micelles and nanoparticles of this invention are distinguished from and exclude liposomes which are composed of bilayers. The micelles of this invention can be composed of either a single monomolecular polymer containing hydrophobic and hydrophilic moieties or an aggregate mixture containing many amphiphilic (i.e. surfactant) molecules formed at or above the critical micelle concentration (CMC), in a polar (i.e. aqueous) solution.

Nanoparticle Carriers

Preferred nanoparticle carriers include the micelles, nanoparticles and dendrimers defined herein, including their pegylated forms and those that contain the amphiphilic molecules defined herein, as well as the proteins, carbohydrates and grafted polymers defined herein. Also included are micelles containing PEG, or poly(ethylene oxide) (PEO), or poly(propylene oxide) (PPO) such as those disclosed by S-F. Chang, et al, in Human Gene Therapy 15, 481-493 (2004), and references therein. Preferred micelles include the micelles and biocleavable micelles including preparation methods disclosed in U.S. Pat. No. 6,835,718 B2 and references therein, which are hereby incorporated into this invention. Said micelles have the desired antibody substance, active agent, chloroquine substances, intercalators, targeting molecules, grafted polymers and other moieties coupled to the micelle through suitable covalent coupling that can include biocleavable linkages defined herein. For instance, a chloroquine substance or other moiety is covalently coupled to a suitable anchor substance such as an amphiphilic molecule or derivative, which is inserted into said micelle containing an antibody substance, during or after synthesis. Alternatively, an antibody substance is covalently coupled to a suitable anchor substance such as an amphiphilic molecule or derivative, which is inserted into said micelle containing a chloroquine substance, during or after synthesis. For instance, a chloroquine substance and an antibody substance are suitably coupled to HSA, which is covalently coupled to a micelle containing maleimido-4-(p-phenylbutyryl) phosphatidylethanolamine, using the heterobifunctional reagent N-succinimidyl-5-acetylthioacetate (SATA). Also, a PEG derivative of phosphatidylethanolamine (PEG-PE) can be included in the micelle.

Micelles are prepared from block copolymers using well known methods. For instance, a suitable method is disclosed by P. L. Soo, et al, in Langmuir 18, 9996-10004 (2002) for polycaprolactone-block-poly(ethylene oxide). A suitable mixture of chloroquine-coupled lipid, antibody substance and the desired block copolymer are prepared in a suitable solvent such as DMF. Micellation is achieved by slowly adding water (2.5%/minute), with constant stirring, until the desired water content is achieved (i.e. 80-99%). The product is purified by exhaustive dialysis against water. The forgoing reference and references therein are hereby incorporated into this invention.

### Nucleic Acids

For the purposes of this invention, “nucleic acids” are defined as a class of active agents that are limited by category to include any pharmaceutical nucleic acids, meaning useful or potentially useful in therapeutic or prophylactic applications in humans, or any other vertebrate animals and in plants. The most preferred nucleic acids defined as pharmaceutical are nucleic acid active agents against viral and other microbial diseases, against cancers, heart diseases, autoimmune diseases, genetic and other diseases or disorders in humans and other vertebrates. Also included are nucleic acid active agents against viral and other microbial diseases in plants. They also include specific DNA sequences used for gene therapy. Preferred nucleic acids for this invention are DNA, RNA and plasmids disclosed in U.S. patent application Ser. No. 11/323,389 and are incorporated herein by reference.

### Targeting or Biorecognition Molecules

For the purposes of this invention, targeting or biorecognition molecules are moieties suitable for pharmaceutical or diagnostic use that bind to the surface or biological site of a specific cell, tissue or organism. The biological site is considered the “target” of the biorecognition molecule or “targeting moiety” that binds to it. In the prior art, certain drugs are “targeted” by coupling them to a targeting molecule that has a specific binding affinity for the cells, tissue or organism that the drug is intended for. For targeting a composition of this invention, a targeting molecule is coupled to any suitable chloroquine substance that also has coupled an antibody substance. Or, a targeting molecule is coupled to any suitable chloroquine substance that includes an antibody substance and a carrier substance coupled to it. Preferred targeting moieties include, but are not limited to, those disclosed by S Jaracz, et al, Bioorg Med. Chem. (2005) 13(17): 5043-54, including references therein. Categories of targeting molecules and biorecognition molecules useful in this invention are described below.

Ligand.

A ligand functions as a type of targeting or biorecognition molecule defined as a selectively bindable material that has a selective (or specific), affinity for another substance. The ligand is recognized and bound by a usually, but not necessarily, larger specific binding body or “binding partner”, or “receptor”. Examples of ligands suitable for targeting are antigens, haptens, biotin, biotin derivatives, lectins, galactosamine and fucosylamine moieties, receptors, substrates, coenzymes and cofactors among others.

When applied to this invention, a ligand includes an antigen or hapten that is capable of being bound by, or to, its corresponding antibody or fraction thereof. Also included are viral antigens, nucleocapsids and cell-binding viral derivatives including those from any DNA and RNA viruses, AIDS, HIV and hepatitis viruses, adenoviruses, adeno-associated viruses (AAV), alphaviruses, arenaviruses, coronaviruses, flaviviruses, herpesviruses, myxoviruses, oncornaviruses, papovaviruses, paramyxoviruses, parvoviruses, picornaviruses, poxviruses, reoviruses, rhabdoviruses, rhinoviruses, togaviruses and viroids; any bacterial antigens including those of gram-negative and gram-positive bacteria, acinetobacter, achromobacter, bacteroides, clostridium, chlamydia, enterobacteria, haemophilus, lactobacillus, neisseria, staphyloccus, and streptoccocus; any fungal antigens including those of aspergillus, candida, coccidiodes, mycoses, phycomycetes, and yeasts; any mycoplasma antigens; any rickettsial antigens; any protozoan antigens; any parasite antigens; any human antigens including those of blood cells, virus infected cells, genetic markers, heart diseases, oncoproteins, plasma proteins, complement factors, rheumatoid factors. Included are cancer and tumor antigens such as alpha-fetoproteins, prostate specific antigen (PSA) and CEA and cancer markers, among others.

Other substances that can function as ligands for targeting are certain vitamins (i.e. folic acid, B₁₂), steroids, prostaglandins, carbohydrates, lipids, antibiotics, drugs, digoxins, pesticides, narcotics, neuro-transmitters, and substances used or modified so that they function as ligands. Ligands also include various substances with selective affinity for receptors that are produced through recombinant DNA, genetic and molecular engineering. Except when stated otherwise, ligands of the instant invention also include the ligands as defined by K. E. Rubenstein, et al, U.S. Pat. No. 3,817,837 (1974).

Also included are any suitable vitamins for targeting such as vitamin B₆ (T. Zhu, et al., (1994) Bioconjugate Chem. 5, 312.). Also included are targeting receptors such as for liver cells using the asialo-glycoprotein receptors (X. M. Lu, et al, (1994) Nucl. Med. 35, 269). Also included are suitable octreotides or octreotate, the carboxylic acid derivative of octreotide for targeting somatostatin receptors, among others. Also included are peptides which bind to integrins and the EGF receptor family.

Targeting Antibody Substance.

When applied to targeting moieties of this invention, one preferred category is an antibody substance, which is defined herein and includes all classes of antibodies, synthetic antibodies and monoclonal antibodies. Also included are antibodies used for specific cell or tissue targeting such as antibodies that bind to specific cell receptors such as anti-transferrin antibodies used to cross the blood brain barrier.

Receptor.

A receptor functions as a type of targeting molecule defined for this invention as a specific binding body or “partner” or “ligator” that is usually, but not necessarily, larger than the ligand it can bind to. For the purposes of this invention, it is a specific substance or material or chemical or “reactant” that is capable of selective affinity binding with a specific ligand.

Under certain conditions, this invention is applicable to using other substances as receptors. For instance, other suitable receptors include naturally occurring receptors, any hemagglutinins and cell membrane that bind specifically to hormones, vitamins, drugs, antibiotics, cancer markers, genetic markers, viruses, bacteria and histocompatibility markers.

Other receptors also include enzymes, especially cell surface enzymes such as neuraminidases. Also included are chalones, cavitands, thyroglobulin, intrinsic factor, chelators, staphylococcal protein A, protein G, bacteriophages, cytochromes and lectins.

Most preferred are certain proteins or protein fragments (i.e. hormones, toxins), and synthetic or natural polypeptides with cell surface affinity such as growth factors that include basic fibroblast growth factors (bFGF). Preferred targeting molecules also include certain proteins and protein fragments or derivatives with affinity for cells, tissues or microorganisms that are produced through recombinant DNA, genetic and molecular engineering.

Blood-Brain Barrier Agents.

A preferred and separate category of substances are blood-brain barrier (BBB) targeting agents. Blood-brain barrier agents are substances that can penetrate the BBB and carry other substances into the brain. There are certain compounds needed for penetrating the BBB, as are disclosed by D. J. Begley, in J. Pharm. Pharmacol. 48, 136-146 (1996) and by W. M. Partridge, et al, in J. Cereb. Blood Flow Metab. 17, 713-731 (1997), and incorporated herein. Such compounds include those which are more lipophilic, are capable of changing to effective chirality after crossing the blood-brain barrier, have side chain moieties which enhance compound transport via blood-brain barrier transporter mechanisms, or are coupled with specific BBB-penetrating antibodies, defined herein.

Transduction Vector.

Transduction vectors are known in the prior art under a wide variety of names. For this invention a transduction vector is defined as a peptide substance suitable for pharmaceutical use that promotes cellular uptake across the cell membrane and may include intracellular transport such as into the cell nucleus. Preferred transduction vectors or “fusion vectors” or “fusion moieties” or “membrane transduction” moieties are certain membrane translocation or membrane transfer peptides that can also include carbohydrates, lipids and polymers and combinations of these substances. Preferred transduction vectors are peptides (“fusion peptides” or “peptide vectors”) including those with “transduction domains” in their amino acid sequence. Preferred transduction vectors have a molecular weight between 1000 and 100,000 Daltons, most preferred between 1200 and 80,000 Daltons. The category of transduction vectors as defined for this invention specifically exclude complex proteins such as antibodies and enzymes. Some preferred transduction vectors for this invention include, but are not limited to, any derived sequences or extracts of any signal peptides or any fusogenic peptides including: TAT (i.e. from HIV virus), herpes simplex virus VP-22, hepatitis B virus PreS2 translocation motif (TLM) and antennapedia homeoproteins (i.e. penetratins). Preferred transduction vectors also include poly arginines (i.e. containing 5 or more, preferably from 6 to 12 arginines and with or without one or more terminal cysteines), poly histidines, poly lysines, poly ornithines and combinations of these amino acids with or without one or more terminal cysteines. Also included are the peptide vectors disclosed by P. M. Fischer, et al, in Reviews Bioconj. Chem 12, 825-841 (2001), C—H Tung, et al, in Reviews Bioconj. Chem. 11, 605 (2000) and references therein. Preferred examples of transduction vectors in this invention are peptide vectors which have been employed for transport of active agents including nucleic acids into cells. Preferred examples include conjugates of a carrier substance with penetratins or signal peptides to increased uptake rates due to the membrane translocation properties of these peptides. In U.S. patent application Ser. No. 10/923,112, Table I. is a list of some peptides that are preferred transduction vectors in this invention. Preferred peptides include; pAntp(43-58) Penetratins, retro-inverso pAntp(43-58) Penetratins, W/R Penetratins, antennapedia peptides, pAntp(52-58), any sequence that includes HIV TAT, or HIV TAT C-terminus peptides, viral fusion peptides, gp41 fusion sequences, gp41 fusion sequence SV40 NLS, CR-gp41 fusion peptides, C. crocodylus Ig(v) light chains, C. crocodylus Ig(v) light chain —SV40 NLS, PreS2-TLM, Transportans, SynB1, MPS (kaposi FGF signal sequences), MPS (human integrin beta3 signal sequences), P3, Model amphiphilic peptides, KALA, hemagglutinin envelope fusion peptides and any arginine containing peptides (R7).

Cell Receptor Binding Peptides.

Preferred targeting moieties are cell receptor binding (CRB) peptides that bind to distinct receptors, which upon binding, may also mediate endocytosis of a CRB peptide-antibody substance conjugate or CRB peptide-ODN complex. Also included are peptides which bind to integrins and to the EGF receptor family. In U.S. patent application Ser. No. 10/923,112, Table II. is a list of some receptor binding peptides that are preferred in this invention. Preferred receptor binding peptides include; Fc receptor binding peptides, antagonists to IGF-1 receptors, beta-endorphin receptor ligands, hepatocyte specific delivery peptides, tuftsin (Thr-Lys-Pro-Arg), and cell fusion and hemolysis inhibitor peptides.

### Cyclodextrins

A cyclodextrin (CD) monomer, is an oligosaccharide of glucose molecules coupled together to form a ring that is conical with a hydrophobic, hollow interior or cavity. Cyclodextrin monomers are one of the starting materials for making grafted polymers as described in the instant invention. They are any cyclodextrin suitable for pharmaceutical use, including alpha-, beta-, and gamma-cyclodextrins, and their combinations, analogs, isomers, and derivatives. In describing this invention, references to a cyclodextrin “complex”, means a noncovalent inclusion complex. An inclusion complex is defined herein as a cyclodextrin functioning as a “host” molecule, combined with one or more “guest” molecules that are contained or bound, wholly or partially, within the hydrophobic cavity of the cyclodextrin or its derivative.

Cyclodextrin Dimers, Trimers and Polymers.

For this invention, a cyclodextrin dimer is a preferred category of cyclodextrin derivative defined as two cyclodextrin molecules covalently coupled or cross-linked together to enable cooperative complexing with a guest molecule. Examples of some CD dimers that are derivatized and used in the drug carriers of this invention, are described by; Breslow, R., et al, Amer. Chem. Soc. 111, 8296-8297 (1989); Breslow, R., et al, Amer. Chem. Soc. 105, 1390 (1983) and Fujita, K., et al, J. Chem. Soc., Chem. Commun., 1277 (1984).

A cyclodextrin trimer is another preferred category of cyclodextrin derivative defined as three cyclodextrin molecules covalently coupled or cross-linked together to enable cooperative complexing with a guest molecule. Another preferred cyclodextrin is a cyclodextrin polymer defined as a unit of more than three cyclodextrin molecules covalently coupled or cross-linked together to enable cooperative complexing with several guest molecules. Also included are the “linear” cyclodextrin polymers disclosed by Davis, et al, U.S. Pat. No. 6,509,323 B1.

For this invention, preferred cyclodextrin dimer, trimer and polymer units are synthesized by covalently coupling through chemical groups such as through coupling agents. The synthesis of preferred cyclodextrin dimer, trimer and polymer units does not include the use of proteins or other “intermediate coupling substances”. Cooperative complexing means that in situations where the guest molecule is large enough, the member cyclodextrins of the CD dimer, trimer or polymer can each noncovalently complex with different parts of the same guest molecule, or with smaller guests, alternately complex with the same guest. An improved cyclodextrin dimer, trimer or polymer comprises combinations of different sized cyclodextrins to synthesize these units. Combinations for this invention can include the covalent coupling of an alpha CD with a beta CD, an alpha CD with a gamma CD, a beta CD with a gamma CD and polymers with various ratios of alpha, beta and gamma cyclodextrins.

Most preferred are cyclodextrin dimers, trimers and polymers containing cyclodextrin derivatives such as carboxymethyl CD, glucosyl CD, maltosyl CD, hydroxypropyl cyclodextrins (HPCD), 2-hydroxypropyl cyclodextrins, 2,3-dihydroxypropyl cyclodextrins (DHPCD), sulfobutylether cyclodextrins (SBECD), ethylated and methylated cyclodextrins.

Also preferred are oxidized cyclodextrin dimers, trimers and polymers that provide aldehydes and any oxidized derivatives that provide aldehydes. Some examples of suitable derivatives are disclosed by Pitha, J., et al, J. Pharm. Sci. 75, 165-167 (1986) and Pitha, J., et al, Int. J. Pharmaceut. 29, 73-82 (1986).

Also preferred are any amphiphilic CD dimers, trimers and polymers made from derivatives such as those disclosed in, but not limited to, those disclosed or referenced in U.S. patent application Ser. No. 11/323,389 and are incorporated herein by reference.

Cyclodextrin Blocks.

A CD-block is a category of carrier substances defined as a CD dimer, trimer or polymer that is used as a component, or unit (i.e. building block) for additional cross linking with other polymer blocks to produce a carrier substance suitable for pharmaceutical use or are coupled to the carrier substances of this invention. Preferred cyclodextrin blocks (CD block) provide for the incorporation of cyclodextrin derivatives into carrier substances that include micelle-forming amphiphilic molecules through copolymerization with other polymer blocks or grafted polymers defined herein. The CD blocks can include CD dimers, CD trimers or CD polymers. The CD blocks can be primarily hydrophilic to produce micelles with CD moieties in the hydrophilic shell. Or, the CD blocks can be primarily hydrophobic to produce micelles with CD moieties in the hydrophobic core. The CD blocks also have available suitable reactive groups that can copolymerize with other block polymers, using suitably modified methods described and referenced by G. S. Kwon, IN: Critical Reviews in Therapeutic drug Carrier Systems, 15(5):481-512 (1998). For example, a CD derivative (i.e. CD dimer) is prepared and made hydrophobic by adding alkyl or aromatic groups (i.e. methylation ethylation, or benzylation), and also has available an N carboxyanhydride (NCA) group coupled through a suitable spacer.

One form of CD block is methylated-CD-CD-poly(aspartate)_{N}-NCA (where N=1-10). This CD block can then be copolymerized with suitable blocks of alpha-methyl-omega-amino-poly(ethylene oxide) (PEO) in suitable solvent (CHCl₃:DMF) to produce a micelle-forming diblock amphiphilic molecule. The resulting diblock is CD-block-PEO. With suitable modifications PEG is used in place of PEO. Also, triblocks such as PEO-block-CD-block-PEO can be prepared and used. Other combinations for the CD-blocks of this invention can include the covalent coupling of an alpha CD with a beta CD, an alpha CD with a gamma CD; a beta CD with a gamma CD and polymers with various ratios of alpha, beta and gamma cyclodextrins. Pendant PEG. Pendant polyethylene glycol is one preferred carrier substance for synthesizing the compositions of this invention suitable for pharmaceutical or diagnostic use. Pendant PEG is defined here as derivatized or δgrafted” with side functional groups or “branches” along the backbone of the molecule. The grafted functional side group can be comprised of propionic acid groups or alkyl chains of 2, 3, 4, 5, 6, or more carbon atoms that terminate in a carboxylic acid, or a primary amine, or an aldehyde, or a thiol, or combinations of these.

Pendant PEG (also called “multi-branched PEG”), is available in a variety of molecular masses and with various numbers of functional groups per molecule. For instance, SunBio USA, Orinda, Calif. 94563, offers PEG in molecular weights of 10, 12, 18, 20, 30, 35 and 100 kilo Daltons (KDa), with 6, 8, 10, 12, 14, 16, 18, or 20 functional side groups or “branches”.

For the present invention, preferred pendant PEG has been disclosed in, but not limited to, those disclosed or referenced in U.S. patent application Ser. No. 11/323,389 and are incorporated herein by reference. Preferred pendant PEG ranges from 2,000 Daltons to 1,000,000 Daltons, most preferably a molecular weight of 20,000 or greater to prevent rapid elimination of the PEG-conjugated composition from the bloodstream.

Targeted Chloroquine-Coupled Carriers.

A targeted chloroquine-coupled carrier is composed of a carrier substance suitable for pharmaceutical use that has chloroquines and a targeting molecule coupled to it such as an antibody substance. The carrier is thereby targeted through the specific binding properties of the targeting molecule coupled to the surface. During the coupling of the targeting molecule, the functions of the targeting molecule, chloroquines and the targeted carrier are not irreversibly or adversely inhibited. Preferably, the targeting molecule maintains specific binding properties that are functionally identical or homologous to those it had before coupling. Preferably, the targeting molecule is coupled through a suitable spacer to avoid steric hindrance. Targeted carriers coupled to avidin and streptavidin are useful for noncovalent coupling to any suitable biotinylated chloroquine substance and antibody substance. Similarly, chloroquines suitably coupled to antibody are noncovalently (antigenically) coupled to another antibody, or to a peptide or other suitable substance that has the appropriate biorecognition properties. Another useful composition comprises protein A, protein G, or any suitable lectin that has been covalently coupled to chloroquines and active agents of this invention.

Capping Moiety.

A capping moiety is defined here as a substance suitable for pharmaceutical use that is used to consume or cap any available reactive groups dr functional groups to prevent further coupling or other reactions on the carrier of this invention. The capping moiety may also provide certain desired properties such as neutral charge, or positive charge or negative charge as desired. The capping moiety may also provide increased water solubility or may provide hydrophobicity. The capping moiety may also provide a type of label for colorimetric or fluorometric detection.

Some preferred examples of capping moieties are ethanol amines, glucose amines, mercaptoethanol, any suitable amino acids, including gylcines, alanines, leucines, phenylalanines, serines, tyrosines, tryptophanes, asparagines, glutamic acids, cysteines, lysines, arginines and histidines, among others. Preferred capping moieties also include suitable halogens (including Br, Cl, I, and F) and fluorophores or dyes.

### Diagrams of Preferred Compositions In This Invention

1. Chloroquine-Coupled Pendant Carrier Substance.

A preferred chloroquine-coupled carrier substance is a carrier substance as defined herein, containing one or more chloroquine substances covalently coupled to said carrier substance. Accordingly, the chloroquine-coupled carrier substance of the present invention is represented by the following formula:

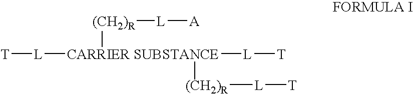

Formula I represents any suitable carrier substance as defined herein that includes a coupled chloroquine substance and coupled moieties as described below. The carrier substance also includes one, two or more branching or pendant units; (CH₂)_{R} covalently coupled to said carrier substance and wherein R is an integer between 1 and 30, preferably between 2 and 10. Also, wherein said pendant unit terminates in either a functional group or is terminally coupled to moieties “L-A” or “L-T” as defined below and wherein said moieties may alternate in their number, sequence and frequency depending on the desired carrier substance used.

In Formula I, A is at least one moiety selected from the group of protein active agents, peptide active agents and CCAs as disclosed herein independently and covalently coupled to the carrier substance through linkage L.

In Formula I, T is independently and covalently coupled to the carrier substance through linkage L. In a preferred embodiment of this invention, T is at least one moiety selected from the group of chloroquine substances as described herein.

In addition to being at least one chloroquine substance, T may also be a member independently selected from the group consisting of hydrogen (H), hydroxyl (OH), halogen, targeting moiety (TM), transduction vector (TV), amphiphilic molecule and capping moiety.

In addition to being at least one chloroquine substance, T may also be a member independently selected from the group consisting of a grafted polymer as disclosed herein that is biocompatible and includes protamines, antibody substance, PEG, HPMA, PEI, PLL, CD, CD dimers, CD trimers and CD polymers. Wherein said grafted polymer is appropriately end capped as is known in the prior art and which also may be substituted with moieties that do not adversely affect the functionality of the grafted polymer for its intended purpose. Also wherein said grafted polymer has a molecular weight range from 500 to 200,000 Daltons, preferably from 1,000 to 50,000 Daltons.

Also wherein T as described herein is coupled to said pendant carrier substance with the proviso that a mixture of chloroquine substances, hydrogens, hydroxyls, targeting moieties, cell transduction vectors, amphiphilic molecules and grafted polymers may be found on the same carrier substance and/or within the same carrier substance composition.

L is a covalent linkage between said carrier substance and substance A or T as defined herein, through functional groups defined herein and may include one or more coupling agents as defined herein. Said linkage L may also include suitable spacer molecules and may be biocleavable as defined herein.

2. Chloroquine-Coupled Carrier Substance.

A preferred chloroquine-coupled carrier substance is a carrier substance as defined herein, without pendant groups and containing one or more chloroquine substances covalently coupled to said carrier substance. Accordingly, the chloroquine-coupled carrier substance of the present invention is represented by the following formula:

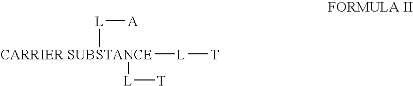

Formula II represents any suitable carrier substance as defined herein that includes a coupled chloroquine substance and coupled moieties as described below. Also, wherein said carrier substance is coupled to moieties “L-A” or “L-T” as defined below and wherein said moieties may alternate in their number, sequence and frequency depending on the desired carrier substance used.

In Formula II, A is at least one moiety selected from the group of protein active agents, peptide active agents and CCAs as disclosed herein independently and covalently coupled to the carrier substance through linkage L.

In Formula II, T is independently and covalently coupled to the carrier substance through linkage L. In a preferred embodiment of this invention, T is at least one moiety selected from the group of chloroquine substances as described herein.

In addition to being at least one chloroquine substance, T may also be a member independently selected from the group consisting of hydrogen (H), hydroxyl (OH), halogen, CCA, targeting moiety (TM), transduction vector (TV), amphiphilic molecule and capping moiety.

In addition to being at least one chloroquine substance, T may also be a member independently selected from the group consisting of a grafted polymer as disclosed herein that is biocompatible and includes protamines, antibody substances, PEG, HPMA, PEI, PLL, CD, CD dimers, CD trimers and CD polymers. Wherein said grafted polymer is suitably end capped as is known in the prior art and which also may be substituted with moieties that do not adversely affect the functionality of the grafted polymer for its intended purpose. Also, wherein said grafted polymer has a molecular weight range from 500 to 200,000 Daltons, preferably from 1,000 to 50,000 Daltons.

Also wherein T as described herein is coupled to said carrier substance with the proviso that a mixture of chloroquine substances, hydrogens, hydroxyls, Protein active agents, peptide active agents and CCAs, targeting moieties, cell transduction vectors, amphiphilic molecules and grafted polymers may be found on the same carrier substance and/or within the same carrier substance composition.

L is a covalent linkage between said carrier substance and substance A or T as defined herein, through functional groups defined herein and may include one or more coupling agents as defined herein. Said linkage L may also include suitable spacer molecules and may be biocleavable as defined herein.

3. Chloroquine-Coupled Noncovalent Carrier Substance.

A preferred chloroquine-coupled carrier substance is a noncovalent carrier substance as defined herein, containing one or more chloroquine substances and one or more protein active agents, peptide active agents or CCAs coupled to said carrier substance wherein at least one said chloroquine substance or CCA or other moiety is coupled noncovalently.

Accordingly, the chloroquine-coupled noncovalent carrier substance of the present invention is represented by the following formula:
(A)_{N}-NONCOVALENT CARRIER SUBSTANCE-L-(T)_{O} FORMULA III

Formula III includes a suitable carrier substance selected from the group of noncovalent coupling proteins (i.e. antibody, streptavidins), protamines, histones, cationic grafted polymers, cationic polymers and cationic lipids as defined herein. Formula III also includes a coupled chloroquine substance and coupled moieties as described below. Also, wherein said carrier substance is coupled to moiety “L-T” as defined below and wherein said moiety may vary in number from 1 to 1000, and vary in sequence and frequency depending on the desired carrier substance used.

In Formula III, A is at least one moiety selected from the group of protein active agents, peptide active agents and CCAs as disclosed herein is independently and noncovalently coupled to the carrier substance through cationic-anionic charge attraction or through avidin-biotin linkage wherein A is an antibody covalently coupled to biotin.

In Formula III, T is independently and covalently coupled to the carrier substance through linkage L. In a preferred embodiment of this invention, T is at least one moiety selected from the group of chloroquine substances as described herein.

In Formula III, N and O may be a number from 1 to 100, preferably from 1 to 10.

Wherein O is more than 1, in addition to being at least one chloroquine substance, T may also be a member independently selected from the group consisting of hydrogen (H), hydroxyl (OH), halogen, CCA, targeting moiety (TM), transduction vector (TV), amphiphilic molecule and capping moiety. Also wherein O is more than 1, in addition to being at least one chloroquine substance, T may be a member independently selected from the group consisting of a grafted polymer as disclosed herein that is biocompatible and includes protamines, antibody substances, PEG, HPMA, PEI, PLL, CD, CD dimers, CD trimers and CD polymers.

Wherein said grafted polymer is suitably end capped as is known in the prior art and which also may be substituted with moieties that do not adversely affect the functionality of the grafted polymer for its intended purpose. Also, wherein said grafted polymer has a molecular weight range from 500 to 200,000 Daltons, preferably from 1,000 to 50,000 Daltons.

Also wherein T as described herein is coupled to said carrier substance with the proviso that a mixture of chloroquine substances, hydrogens, hydroxyls, Protein active agents, peptide active agents and CCAs, targeting moieties, cell transduction vectors, amphiphilic molecules and grafted polymers may be found on the same carrier substance and/or within the same carrier substance composition.

L is a covalent linkage between said carrier substance and substance T as defined herein, through functional groups defined herein and may include one or more coupling agents as defined herein. Said linkage L may also include suitable spacer molecules and may be biocleavable as defined herein.

4. Chloroquine-Coupled CCA.

A preferred chloroquine-coupled CCA is one or more chloroquine substances covalently coupled to one or more protein active agents, peptide active agents or CCAs. Accordingly, the chloroquine-coupled CCA of the present invention is represented by the following formula:
(A)_{N}-L-(T)_{O} FORMULA IV

In Formula IV, A is at least one moiety selected from the group of protein active agents, peptide active agents and CCAs as disclosed herein.

In Formula IV, T is at least one moiety selected from the group of chloroquine substances as described herein and wherein T is covalently coupled to A through linkage L.

In Formula IV, N and O may be a number from 1 to 100, preferably from 1 to 10.

Wherein O is more than 1, in addition to being at least one chloroquine substance, T may also be a member independently selected from the group consisting of hydrogen (H), hydroxyl (OH), halogen, CCA, targeting moiety (TM), transduction vector (TV), amphiphilic molecule and capping moiety. Also wherein O is more than 1, in addition to being at least one chloroquine substance, T may be a member independently selected from the group consisting of a grafted polymer as disclosed herein that is biocompatible and includes protamines, antibody substances, PEG, HPMA, PEI, PLL, CD, CD dimers, CD trimers and CD polymers. Wherein said grafted polymer is appropriately end capped as is known in the prior art and which also may be substituted with moieties that do not adversely affect the functionality of the grafted polymer for its intended purpose. Also wherein said grafted polymer has a molecular weight range from 500 to 200,000 Daltons, preferably from 1,000 to 50,000 Daltons.

L is a covalent linkage between said substance A and substance T as defined herein, through functional groups defined herein and may include one or more coupling agents as defined herein. Said linkage L may also include suitable spacer molecules and may be biocleavable as defined herein.

### EXAMPLES

In the examples herein, percentages are by weight unless indicated otherwise. During the synthesis of the compositions of the instant invention, it will be understood by those skilled in the art of organic synthesis, that there are certain limitations and conditions as to what compositions will comprise a polymer carrier suitable for pharmaceutical use and may therefore be prepared mutatis mutandis. It will also be understood in the art of chloroquines, protein or peptide active agents, antibody substances and nucleic acids that there are limitations as to which derivatives and/or coupling agents can be used to fulfill their intended function.

The terms “suitable” and “appropriate” refer to derivatives and synthesis methods known to those skilled in the art for performing the described reaction or other procedure. In the references to follow, the methods are hereby incorporated herein by reference. For example, organic synthesis reactions, including cited references therein, that are useful in the instant invention are described in “The Merck Index”, 9, pages ONR-1 to ONR-98, Merck & Co., Rahway, N.J. (1976), and suitable protective methods are described by T. W. Greene, “Protective Groups in Organic Synthesis”, Wiley-Interscience, NY (1981), among others.

All reagents and substances listed, unless noted otherwise, are commercially available from Applied Biosystems Division, Perkin-Elmer; Aldrich Chemical Co., WI 53233; Sigma Chemical Co., Mo. 63178; Pierce Chemical Co., IL. 61105; Eastman Kodak Co., Rochester, N.Y.; Pharmatec Inc., Alachua Fla. 32615; and Research Organics, Cleveland, Ohio. Or, the substances are available or can be synthesized through referenced methods, including “The Merck Index”, 9, Merck & Co., Rahway, N.J. (1976). Additional references cited in U.S. Pat. No. 6,048,736 and PCT/US99/30820, are hereby incorporated herein by reference.

### Derivatized Carriers Substances

For this invention, the general synthesis approach is; (1) produce or modify or protect, as needed, one or more functional groups on a chloroquine substance and (2) using one or more coupling methods, couple a chloroquine substance to a protein CCA or peptide CCA directly or through a carrier substance suitable for pharmaceutical use.

Also, as described below, the carrier may be suitably derivatized to include other useful substances and/or chemical groups (e.g. targeting molecules), to perform a particular function. Depending on the requirements for chemical synthesis, the derivatization are done before coupling the chloroquine substance, or afterward, using suitable protection and deprotection methods as needed.

The carrier substance is suitably derivatized and coupled through well-known procedures used for available amino, sulfhydryl, hydroxyl, or vinyl groups. Also, for certain carbohydrates added to the carrier substance, vicinal hydroxyl groups can be appropriately oxidized to produce aldehydes. Any functional group can be suitably added through well-known methods while preserving the carrier substance structure and properties. Examples are: amination, esterification, acylation, N-alkylation, allylation, ethynylation, oxidation, halogenation, hydrolysis, reactions with anhydrides, or hydrazines and other amines, including the formation of acetals, aldehydes, amides, imides, carbonyls, esters, isopropylidenes, nitrenes, osazones, oximes, propargyls, sulfonates, sulfonyls, sulfonamides, nitrates, carbonates, metal salts, hydrazones, glycosones, mercaptals, and suitable combinations of these. The functional groups are then available for suitable coupling or cross-linking using a bifunctional reagent.

Suitable coupling or cross-linking agents for preparing the carriers of the instant invention are a variety of coupling reagents, including oxiranes (epoxides) previously described. Also useful are methods employing acrylic esters such as m-nitrophenyl acrylates, and hexamethylene diamine and p-xylylenediamine complexes, and aldehydes, ketones, alkyl halides, acyl halides, silicon halides and isothiocyanates.

Synthesis Materials.

All chemicals were reagent grade and are available from Acros Organics/Fisher Scientific, Pittsburgh, Pa.; Alltech Assoc., Deerfield, Ill.; Amersham Pharmacia Biotech, Piscataway, N.J.; Calbiochem, San Diego, Calif.; Molecular Probes, Eugene, Oreg.; Promega Corp., Madison, Wis.; Sigma-Aldrich, St. Louis, Mo. 63178; TCI America, Portland Oreg.; or VWR International, West Chester, Pa. 19380. Deionized water is used where not stated otherwise.

Some reagents used and their abbreviations are; benzotriazol-1-yloxy-tris(dimethylamino)-phosphonium hexafluorophosphate (BOP), 1-Decene, n-butylamine, 2,2,2-trifluoroethanol, dicyclohexyl carbodiimide (DCC), 1,3-diisopropyl carbodiimide (DIC), N-(3-dimethylaminopropyl)-N′-ethylcarbodiimide (EDC), ethylenediamine tetraacetic acid (EDTA), 3-nitrophenol, fluorescein isothiocyanate (FITC), fluorenyl methoxycarbonyl (Fmoc), N-hydroxysuccinimide (NHS), ethanethiol, n-butylamine, 4-(dimethylamino)-pyridine (DMAP), dithiothreitol (DTT), 1,1,2-trichloroethane (TCE), trifluoroacetic acid (TFA), trityl (Trt) and sodium dodecyl sulfate (SDS). Some suitable solvents are; ethyl acetate (EtOAc), methanol (MeTOH), tetrahydrofuran (THF), N,N-dimethyl formamide (DMF), dimethyl sulfoxide (DMSO), isopropanol and n-heptane. Phosphate-buffered saline (PBS) is 0.01 M sodium phosphate and 0.015 M sodium chloride pH about 7.2 or adjusted with 0.1 M HCl, 0.1 M KOH (or NaOH) solution as needed.

Testing Procedures.

The chloroquine or chloroquine derivative concentration in the preparations was determined by absorbance or by fluorescence using 485 nm excitation wavelength and reading at 528 nm emission wavelength. The sample concentration was determined by using least squares (linear regression) calculation of the slope and intercept from a standard curve of known concentrations.

Aldehyde concentration in the preparations was determined using the fluorescent indicator, 4′-Hydrazino-2-Stilbazole Dichloride (HSD) based on the method of S. Mizutani, et al, in Chem. Pharm. Bull. 17, 2340-2348 (1969). The sample concentration was determined by using least squares calculation as described previously.

Amine concentration was measured by the following calorimetric test. To 0.02 mL of amine sample in water was added 0.05 mL of borate buffer, pH 8.5. Then 0.05 mL of 0.075% 2,4,6-trinitrobenzene sulfonate (TNBS) was added and mixed. After 20 minutes at rt, the absorbance was read at 420 nm. The absorbance was compared to a glycine standard curve to calculate the sample amine concentration by least squares as described previously.

Carbohydrate concentration was measured by the following colorimetric test. To 0.02 mL of carbohydrate sample in water was added 0.01 mL of 1.5% naphthol in MetOH. Then 0.1 mL of concentrated sulfuric acid was added rapidly to mix. After 20 minutes at rt, the absorbance was read at 620 nm. The absorbance was compared to a dextran or CD standard curve to calculate the sample concentration by least squares as described previously.

Thiol concentration was measured by combining: 0.008 ml of sample and 0.1 ml of 0.0125% 2,2′-dithio-bis(5-nitropyridine) (DTNP) in 62.5% isopropanol, pH 6 to produce a color reaction. The absorbance was read at 405 nm and sample thiol concentration was calculated by linear regression using values from a cysteine standard curve.

### Synthesis of Activated Chloroquine Substances, CCAs and Carrier Substances

The following are methods for synthesizing the compositions of this invention. They are based on J. T. C. Wojtyk, et al, in Langmuir 18, 6081 (2002), for derivatizing a carboxylate group on any suitable carrier substance to provide an activated ester for coupling to a primary amine on a chloroquine substance or any suitable moiety. Conversely, these methods are used under suitable conditions for derivatizing a carboxylate group on any suitable chloroquine substance or antibody including protein or peptide CCAs disclosed herein, to provide an activated chloroquine substance ester for coupling to a primary amine or thiol on any suitable amine- or thiol-containing substance including carrier substances, or other CCA, as defined herein.

If needed, a carrier substance or CCA with a hydroxyl or amino group such as protein, dextran, cyclodextrin or PEG is first derivatized to provide a carboxylated carrier substance by reacting it with acetic (or succinic) anhydride in anhydrous solvent such as DMF. If desired, any suitable carbodiimide can be substituted for DIC such as DCC or EDC.

A. Synthesis of 3-Nitrophenyl Activated Chloroquines, CCAs and Carrier Substances.

A1. 3-Nitrophenyl Activated Carrier. In a 100 mL round-bottom flask equipped with a magnetic stirrer and a nitrogen inlet is placed the carboxylated carrier substance such as pendant PEG with about 15 acid groups (1.00 g, 0.75 mmol acid) and 3-nitrophenol (0.14 g, 1.0 mmol). The mixture is dissolved in 10 mL of dry THF and cooled to 0° C. before a 10 mL THF solution of DIC (0.13 g, 1.0 mmol) and DMAP (0.012 g, 0.10 mmol) is added drop wise via a syringe over a 10 min period. The mixture is allowed to warm gradually to room temperature and stirred at this temperature for 18 h. The urea byproduct is filtered and the filtrate is precipitated from isopropanol to recover the product.

A2. 3-Nitrophenyl Activated Chloroquine Substance or CCA. In a suitable flask, about 0.10 mmol acid equivalents of the carboxylated chloroquine substance (i.e. chloroquine succinate) or carboxylated CCA is combined with about 1.0 mmol of 3-nitrophenol in suitable dry solvent such as THF or DMF, cooled to 0° C. Then about 10 mL of solvent containing 1.0 mmol of DIC is added dropwise. The mixture is stirred 18 hours at rt. The urea byproduct is filtered off and the 3-nitrophenyl activated chloroquine substance ester (i.e. chloroquine ONp) is recovered by precipitation from the filtrate, or isolated by chromatography.

B. Synthesis of N-Hydroxy Succinimidyl (NHS) Activated Chloroquines. CCAs and Carrier Substances.

B1. NHS Activated Carrier. In a 100 mL round-bottom flask equipped with a magnetic stirrer and a nitrogen inlet is placed the carboxylated carrier substance such as pendant PEG with about 15 acid groups (1.00 g, 0.75 mmol acid) and N-hydroxysuccinimide (NHS, 0.12 g, 1.00 mmol). The mixture is dissolved in 5 mL of dry DMF and cooled to 0° C. before a 5 mL DMF solution of DIC (0.13 g, 1.0 mmol) and DMAP (0.012 g, 0.10 mmol) is added drop wise via a syringe over a 10 min period. The mixture is allowed to warm to room temperature and stirred for 18 h at this temperature. The urea byproduct is filtered off and the filtrate is precipitated from isopropanol to recover the product.

B2. NHS Activated Chloroquine Substance or CCA. In a suitable flask, about 0.10 mmol acid equivalents of the carboxylated chloroquine substance (i.e. chloroquine succinate) or carboxylated CCA is combined with about 1.0 mmol of N-hydroxysuccinimide (NHS) in suitable dry solvent such as THF or DMF, cooled to 0° C. Then about 10 mL of solvent containing 1.0 mmol of DIC is added dropwise. The mixture is stirred 18 hours at rt. The urea byproduct is filtered off and the N-hydroxysuccinimide activated chloroquine substance ester (i.e. chloroquine NHS) is recovered by precipitation from the filtrate, or isolated by chromatography.

C. Synthesis of S-Ethyl Activated Chloroquines. CCAs and Carrier Substances.

C1. S-Ethyl Activated Carrier. In a 100 mL round-bottom flask equipped with a magnetic stirrer and a nitrogen inlet is placed the carboxylated carrier substance such as pendant PEG with about 15 acid groups (1.00 g, 0.75 mmol acid) and ethanethiol (0.06 g, 1.00 mmol). The mixture is dissolved in 10 mL of dry THF and cooled to 0° C. before a 10 mL THF solution of DIC (0.13 g, 1.0 mmol) and DMAP (0.012 g, 0.10 mmol) is added drop wise via a syringe over a 10 min period. The mixture is stirred for 18 h at 0° C. The urea byproduct is filtered off and the filtrate is precipitated from isopropanol to recover the product.

C2. S-Ethyl Activated Chloroquine Substance or CCA. In a suitable flask, about 0.10 mmol acid equivalents of the carboxylated chloroquine substance (i.e. chloroquine succinate) or carboxylated CCA is combined with about 1.0 mmol of ethanethiol in suitable dry solvent such as THF or DMF, cooled to 0° C. Then about 10 mL of solvent containing 1.0 mmol of DIC is added dropwise. The mixture is stirred 18 hours at rt. The urea byproduct is filtered off and the S-ethyl activated chloroquine substance ester (i.e. chloroquine S-ethyl ester) is recovered by precipitation from the filtrate, or isolated by chromatography.

D. Activated Ester Chloroquine Substance or Other Moiety.

With suitable modifications, the procedures used to add activated esters to the carboxylated carrier substances described previously, can also be used to add activated esters to carboxylated substances. If needed, a chloroquine substance or other moiety with a hydroxyl or amino group is first carboxylated by reacting it with acetic anhydride in anhydrous solvent such as DMF. These carboxylated substances are then coupled to amino-derivatized carrier substances using carbodiimide.

E. Coupling an Activated Chloroquine Substance to Amino-Containing Substances.

This procedure is used to conjugate amino-containing substances (i.e. any suitable peptide, protein, insulin, antibody, HSA, amino-PEG or amino-dextran) with any suitable activated ester moiety including CCAs and chloroquines that have active ester (i.e. NHS) or isothiocyanate functional groups. At pH 9, conjugation occurs virtually exclusively at the amino group. About 0.2 mmoles of amino-containing substance (i.e., with about 0.1-0.2 mmoles of free primary amines) is dissolved in 1-2 mL of sterile distilled water. To this solution is added 0.1-0.2 mL of 10× conjugation buffer (1M NaHCO₃/Na₂CO₃, pH 9).

A 10 mg/mL DMF solution is freshly prepared of the activated chloroquine or other moiety containing active ester or isothiocyanate. To the buffered carrier solution is added 0.2-0.4 mL of the DMF solution, mixed and allowed to stand at least 2 hours or overnight.

The reaction mixture is purified on a column of Sephadex G-25 in water to remove the excess moiety.

### Addition of Aldehyde Groups Using Glycidol

Carrier substances, chloroquine substances and any other suitable moiety that contains a hydroxyl, amino or sulfhydryl reactive group are derivatized to provide an aldehyde functional group using this method. The substance is first derivatized by coupling glycidol (2, 3 epoxy propanol) to the reactive group. The ether bond coupled glycidol produces a “dihydroxy propyl” moiety (with two terminal, vicinal hydroxyl groups). Then the vicinal hydroxyl groups are oxidized and cleaved with sodium periodate, leaving a terminal aldehyde group.

To an aqueous or nonaqueous solution of the substance to be derivatized is added glycidol at any desired molar ratio. For instance, to 100 mL of 1 mM NaOH in water (pH 8), containing about 8 gm of dissolved dextran 40 (TCI America), average mw 40,000 Daltons (40 kDa), was added 0.34 mL of glycidol (mw 74.02, 96%), mixed and put in the dark at rt for several days (CD159). The resulting dextran-glycidol preparation was concentrated by evaporation over boiling water to about 70 mL, giving a clear solution. The dextran-glycidol preparation was oxidized by adding about 0.94 gm of NaIO₄ in 10 mL of water, mixed and put in the dark at rt for about 1 hour. The resulting dextran-aldehyde was exhaustively dialyzed against water in suitable cellulose tubing (molecular weight cut-off of 12-14 kDa, Spectrum), for 3 days. The dextran-aldehyde dialysate was concentrated by evaporation to about 28 mL.

Alternatively, certain polysaccharides such as inulin or CD, are oxidized without glycidol treatment to produce aldehydes. In any case, the aldehyde product, such as oxidized inulin or CD, is collected by several precipitations with about 5 volumes of 100% isopropanol and cooling to −20° C. for several hours. The precipitate is collected by centrifugation and dissolved in water. Also, it can be further purified by Sephadex™ G50 size exclusion gel chromatography in water or water/MetOH (50%).

The product dry weight was 0.265 gm/mL, determined from drying a 0.10 mL aliquot to constant weight. Dextran (or inulin, CD) concentration is measured as carbohydrate as described herein. Aldehyde concentration is determined using HSD as described previously.

### Amination Methods

Carrier substances, CCAs and chloroquine substances that do not normally contain amino groups are suitably aminated to provide them by methods well known in the art as is disclosed for CD derivatives by A. R. Khan, et al, in Chem. Rev. 98, 1977-1996 (1998) and references therein which are hereby incorporated. For instance, carrier substances such as carbohydrates including inulins, dextrans and cyclodextrins, as well as PEG and other hydroxiated polymers with available hydroxyl groups are readily aminated through tosylation. The hydroxyl groups are first reacted with p-toluene sulfonyl chloride, in suitable anhydrous solvent. Then the tosylate on the reactive site is displaced by treatment with excess sodium azide. Finally, the azide is reduced to an amine with an appropriate hydrogenation method such as with hydrogen and a noble metal catalyst to provide an amino-containing carrier substance.

### Thiolation and Coupling Methods

On amino-containing carrier substances, chloroquine substances and other moieties, the hydrazine or other amino groups are thiolated to provide thiols for disulfide coupling such as between any suitable thiolated carrier substance and thiolated chloroquine substance or protein or peptide CCA. Suitable methods using SPDP or 2-iminothiolane are disclosed by E. J. Wawrzynczak, et al, in C. W. Vogel (ed.) “Immunoconjugates; Antibody Conjugates in Radioimaging and Therapy of Cancer.” NY; Oxford Univ. Press, pp 28-55, (1987).

For instance, primary amino groups on a chloroquine substance (i.e. primaquine, HQ-amine, etc.) or insulin or carrier substance are thiolated in PBS, pH 7.5 by adding a 2× molar excess of SPDP in EtOH and letting it react for about 1 hour at rt. Excess SPDP is removed by size exclusion gel chromatography. Before coupling, the pyridine-2-thione is released by adding a molar excess of DTT to provide sulfhydryl groups.

Preferred thiol coupling in this invention also includes the use of maleimide linkers that include, but are not limited to, those disclosed by EJF Prodhomme in; Bioconjugate Chem., Vol. 14, No. 6, (2003).

Thiol-Disulfide Interchange. This is a method of this invention for coupling two thiolated moieties through their sulfhydryl groups to produce a disulfide linkage. For example, a thiolated carrier substance, thiolated protein or peptide CCA or chloroquine substance is first activated by reacting the sulfhydryls with a slight molar excess of 2,2′-dipyridyl disulfide (2DD), in suitable buffer (i.e. 0.1 M NaHCO₃, pH 8), for about 30 minutes. Depending on the type of substance, the excess 2DD is removed by precipitation or gel exclusion chromatography. The 2DD-activated carrier substance or chloroquine substance is then combined with any suitable thiolated moiety in pH 8 buffer and reacted for 12-24 hours. The substance with disulfide coupled moiety is collected by precipitation or chromatography as before.

Preparation I-A.

Hydroxychloroquine Aldehyde (HQ-Ald) Using Glycidol.

The purpose is to prepare an activated chloroquine substance comprising an aldehyde derivative of a chloroquine substance. (N42) To 4.33 grams (10 millimoles) of hydroxychloroquine (HQ) sulfate (Acros, 98%), dissolved in 25 mL of water was added about 3 mL of 0.1 N NaOH to adjust the pH to about 7.3. To this solution was added about 3.1 mL of glycidol (Sigma-Aldrich, 96%), for about a 4× molar excess of glycidol. The solution was mixed and put in the dark at room temperature (rt) for 48 hours or more to allow coupling of the glycidol to the hydroxyl groups.

The hydroxychloroquine-glycidol product was isolated by splitting the solution into 4 aliquots and diluting with about 6 volumes of isopropanol. The mixtures were placed in a −20° C. for several hours to allow precipitation, then centrifuged 30 minutes at about 2500 rpm. The pellets were dissolved in about 5 mL of water, pooled and precipitated as before, then dissolved in a final volume of 9.5 mL water.

The hydroxychloroquine-glycidol preparation was oxidized by adding 0.10 mL of about 0.16% NaIO₄ in water, mixed and left in the dark at rt for about 25 minutes to produce aldehyde groups. The resulting hydroxychloroquine-aldehyde (HQ-Ald) preparation was collected by repeated (2-3×), precipitations with isopropanol as described. HQ concentration was determined by fluorescence and aldehyde concentration was determined using HSD as described previously. Alternatively, the product is purified by Sephadex™ G50 size exclusion gel chromatography in water and concentrated by evaporation.

If desired, the coupled product is tested for purity using HPLC with an Xterra C₁₈ column (Waters Corp., Chicago Ill.) and a mobile phase of 15% acetonitrile in 25 mM ammonium formate, pH 6.5, flow rate 1 mL per minute. Purity is indicated by characteristic retention times when monitored by absorbance scanning at 300-360 nm and by refractive index.

Alternatively, other hydroxylated chloroquine analogs or amino-containing chloroquine substances are substituted for the hydroxychloroquine. For instance, with suitable modifications, mefloquine (MQ) is substituted for hydroxychloroquine in the above reaction to produce mefloquine-aldehyde (MQ-Ald) or, primaquine is substituted for hydroxychloroquine in the above reaction to produce primaquine-aldehyde (PQ-Ald).

Preparation I-B.

Hydroxychloroquine Succinate (HQ-Suc).

The purpose is to prepare a carboxylated derivative of a chloroquine substance for subsequent coupling (i.e. an ester linkage) to any suitable antibody that can include a carrier substance. Under suitable conditions, any hydroxylated chloroquine substance or amino-containing chloroquine substance can be reacted with a suitable carboxylic acid anhydride including but not limited to acetic, glutaric, succinic, phthalic and maleic anhydrides.

To 10 millimoles of hydroxychloroquine (HQ) dissolved in about 250 mL of anhydrous solvent (i.e. pyridine) containing 0.25 millimoles 4-(dimethylamino)-pyridine (DMAP) and about 50 mg of Na₂SO₄ is added about 15 millimoles of succinic anhydride. The solution is mixed and put in the dark at room temperature (rt) for 6 hours or more.

The slurry is filtered and the hydroxychloroquine-succinate (HQ-Suc, or chloroquine succinate) product is isolated by precipitation in suitable solvent at −20° C., then centrifuged 30 minutes at about 2500 rpm. HQ-Succ concentration is determined by absorbance (300-360 nm) or fluorescence.

Alternatively, the product is purified by Sephadex™ G10 size exclusion gel chromatography in 50% methanol/water and concentrated by evaporation. If desired, the coupled product is tested for purity using HPLC with an Xterra C₁₈ column (Waters Corp., Chicago Ill.) and a mobile phase of 15% acetonitrile in 25 mM ammonium formate, pH 6.5, flow rate 1 mL per minute.

Alternatively, other hydroxylated chloroquine analogs or amino-containing chloroquine substances are substituted for the hydroxychloroquine. For instance, with suitable modifications, mefloquine (MQ) is substituted for hydroxychloroquine in the above reaction to produce mefloquine-succinate (MQ-Suc) or, primaquine (PQ) is substituted for hydroxychloroquine in the above reaction to produce primaquine-succinate (PQ-Suc).

3-Nitrophenyl, N-Hydroxysuccinimidyl and S-Ethyl Activated Ester Chloroquine Substances.

Any carboxylated chloroquine substances described herein are suitably derivatized to produce the activated ester form of a chloroquine substance including but not limited to 3-nitrophenyl, N-hydroxysuccinimidyl and S-ethyl activation, described herein. The procedures are suitably modified for using DIC and DMAP and suitable solvents. The mixture is allowed to warm gradually to room temperature and stirred at this temperature for 18 h. The urea byproduct is filtered and the product is precipitated from suitable solvent to recover the product.

Preparation II.

Hydroxychloroquine Amine Using Epoxypropylphthalimide.

(N43) To 2.16 grams (5 millimoles) of hydroxychloroquine (HQ) sulfate (Acros, 98%), dissolved in 8 mL of water (pH 5), was added about 0.2 mL of 1 N NaOH to adjust the pH to about 6.5. To this solution was added about 25 mL of N-(2,3-epoxypropyl)phthalimide (EPP, Sigma-Aldrich, 98%), in 80% DMF/water, for about a 2× molar excess. The solution was mixed and put in the dark at room temperature (rt) for 48 hours or more to allow coupling of the EPP to the hydroxyl groups.

To remove the phthalate by hydrolysis, the pH was adjusted to about 9 with about 3 mL of 1 N NaOH. Then about 0.8 mL (2× molar excess) of hydrazine hydrate (64%, fw 50.06) was added, mixed and put in the dark at rt for 48 hours or more. The reaction mixture was then concentrated by evaporation. The hydroxychloroquine amine product was purified by Sephadex™ G15 size exclusion gel chromatography in 50% MetOH/water and concentrated by evaporation under N₂. HQ concentration was determined by fluorescence and amine concentration was determined using TNBS as described previously.

Alternatively, other hydroxylated chloroquine analogs or chloroquine substances are substituted for the hydroxychloroquine. For instance, with suitable modifications, mefloquine (MQ) is substituted for hydroxychloroquine in the above reaction to produce mefloquine amine.

Hydroxychloroquine-Hydrazine. In another preferred hydroxychloroquine amine embodiment, hydroxychloroquine-aldehyde, disclosed herein, is coupled to excess hydrazine in water, to provide hydroxychloroquine-hydrazine with a biocleavable hydrazone linkage.

With suitable modifications, mefloquine-aldehyde is substituted for hydroxychloroquine-aldehyde in the above reaction to produce mefloquine hydrazine.

Quinacrine-Amine. In another preferred embodiment, quinacrine is sulfhydryl- or amino-derivatized, wherein any suitable diamino compound, including hydrazine are suitably coupled to quinacrine. For instance, to a solution of hydrazine (30 micromoles) in 4 mL of suitable solvent and/or aqueous buffer (i.e. 10 mM Hepes and 1 mM EDTA, pH 7.2), is added about 10 micromole of quinacrine mustard (Sigma-Aldrich) in 2 mL of solvent. The solution is mixed and left at rt in the dark for about 2 hours. The resulting product, quinacrine-coupled hydrazine, is purified by precipitation or by Sephadex™ gel exclusion chromatography. The quinacrine-hydrazine can then be coupled to any suitable carrier substance, or protein or peptide active agent to produce a biocleavable hydrazone linkage.

Alternatively, a dimercapto compound such as dithiothreitol, is coupled to quinacrine mustard in place of a diamino compound, then coupled to any suitable carrier substance or protein or peptide active agent, through thiol-disulfide interchange as disclosed herein, to produce a biocleavable disulfide linkage.

Preparation III-A.

Activated Chloroquine Substance Anhydride.

A1. Chloroquine Anhydrides. The purpose is to prepare an activated chloroquine substance comprising an anhydride derivative of a chloroquine substance for subsequent coupling (i.e. an ester linkage) to any suitable antibody or carrier substance. Under suitable conditions, any chloroquine substance containing an available hydroxyl, thiol or amino functional group is coupled directly to the available amino group on aspartic acid or glutamic acid using a suitable cross linking agent disclosed herein. The resulting chloroquine substance-N-substituted dicarboxylic acid is then converted to an anhydride.

In a preferred embodiment, the chloroquine substance is coupled directly to hydroxylphthalic anhydride in suitable dry solvent using a suitable cross linking agent disclosed herein (i.e. diepoxy butane), in equimolar ratios. The resulting chloroquine-substituted phthalic anhydride is collected by precipitation and/or purified by chromatography.

In another preferred embodiment, the chloroquine substance is first derivatized to give the corresponding active ester (i.e. NHS, ONp or S-ethyl) as described herein. The resulting chloroquine substance active ester is then covalently coupled to the available amino group on aspartic acid or glutamic acid. Based on the methods of M. J. Mardle, et al, J. Chem. Soc. (C), (1968), 237, among others, a suitable N-substituted dicarboxylate is dehydrated in the presence of a dehydrating agent such as acetic anhydride or acyl chloride, among others, to produce the anhydride.

For example, about 10 mmoles of the activated NHS ester of hydroxychloroquine is combined with about 8 mmoles of aspartic acid in suitable dry solvent such as DMF or pyridine and allowed to react for several hours. The resulting chloroquine-N-substituted aspartic acid is collected by precipitation and/or purified by chromatography. In suitable dry solvent, about 5 mmoles of the chloroquine-N-substituted aspartic acid is combined with a slight molar excess of acetic anhydride in a boiling flask. The mixture is heated with refluxing several hours and the resulting chloroquine-N-substituted aspartic anhydride (substituted succinic anhydride) is collected by precipitation and/or purified by chromatography.

Under suitable conditions, aspartic acid is substituted with glutamate or 4-amino phthalate to produce the corresponding chloroquine-N-substituted glutamic anhydride or chloroquine-N-substituted phthalic anhydride.

Under suitable conditions, hydroxychloroquine NHS ester is substituted with suitably protected esters of amodiaquin, amopyroquine, halofantrine, mefloquine, nivaquine, primaquine or tafenoquine to produce the corresponding anhydride.

A2. Peptide CCA Anhydrides. The purpose is to prepare an anhydride derivative of an antibody substance, insulin or other peptide CCA disclosed herein, for subsequent coupling (i.e. an ester linkage) to any suitable chloroquine substance with or without carrier substance. In these methods, protection of certain amino groups (i.e. Fmoc) or sulfhydryls (i.e. Trt) can be done before the reaction and then deprotected afterward, using well known methods. Under suitable conditions, any protein or peptide CCA containing an available hydroxyl, thiol or amino functional group is coupled directly to the available amino group on aspartic acid or glutamic acid using a suitable cross linking agent disclosed herein. The resulting CCA-N-substituted dicarboxylic acid is then converted to an anhydride.

In a preferred embodiment, the CCA is coupled directly to hydroxylphthalic anhydride in suitable dry solvent using a suitable cross linking agent disclosed herein (i.e. diepoxy butane), in equimolar ratios. The resulting CCA-substituted phthalic anhydride is collected by precipitation and/or purified by chromatography.

In another preferred embodiment, the CCA is first derivatized to give the corresponding active ester (i.e. NHS, ONp or S-ethyl) as described herein. The resulting CCA active ester is then covalently coupled to the available amino group on aspartic acid or glutamic acid. Based on the methods of M. J. Mardle, et al, J. Chem. Soc. (C), (1968), 237, among others, a suitable N-substituted dicarboxylate is dehydrated in the presence of a dehydrating agent such as acetic anhydride or acyl chloride, among others, to produce the anhydride.

For example, about 10 mmoles of the activated NHS ester of suitably protected protein or peptide active agent is combined with about 8 mmoles of aspartic acid in suitable dry solvent such as DMF or pyridine and allowed to react for several hours. The resulting peptide-N-substituted aspartic acid is collected by precipitation and/or purified by chromatography. In suitable dry solvent, about 5 mmoles of the peptide-N-substituted aspartic acid is combined with a slight molar excess of acetic anhydride in a boiling flask. The mixture is heated with refluxing several hours and the resulting peptide-N-substituted aspartic anhydride (substituted succinic anhydride) is collected by precipitation and/or purified by chromatography.

Under suitable conditions, aspartic acid is substituted with glutamate or 4-amino phthalate to produce the corresponding peptide-N-substituted glutamic anhydride or peptide-N-substituted phthalic anhydride.

Preparation III-B.

Activated Chloroquine Substance Epoxides.

Chloroquine Epoxides. The purpose is to prepare an activated chloroquine substance comprising an epoxide derivative of a chloroquine substance for subsequent coupling to any suitable antibody, protein or peptide active agent, or carrier substance. Under suitable conditions, any chloroquine substance containing an available hydroxyl, thiol or amino functional group is coupled directly to a suitable vinyl compound using a suitable cross linking agent disclosed herein. The resulting chloroquine substance-vinyl compound is then converted to an epoxide using any suitable peroxy acid. In these methods, protection of certain amino groups (i.e. Fmoc) or sulfhydryls (i.e. Trt) can be done before the reaction and then deprotected afterward, using well known methods.

For example, about 10 mmoles of suitably protected hydroxychloroquine is combined with about 15 mmoles of 3,4-epoxy-1-butene (or 1,2-epoxy-5-hexene) in suitable dry solvent such as DMF or pyridine and allowed to react for several hours. The resulting chloroquine-vinyl product is collected by precipitation and/or purified by chromatography. In suitable dry solvent, about 5 mmoles of the chloroquine-vinyl product is combined with a slight molar excess of peroxyacetic acid (or perbenzoate) in a flask. The mixture is reacted several hours and the resulting chloroquine-epoxide is collected by precipitation and/or purified by chromatography. Under suitable conditions, hydroxychloroquine is substituted with suitably protected amodiaquin, amopyroquine, halofantrine, mefloquine, nivaquine, primaquine or tafenoquine to produce the corresponding peroxide.

Preparation IV-A.

Biocleavable Primaquine-Coupled Combinative Agents.

(N45) The chloroquine substance, primaquine (PQ), is derivatized with a bifunctional, amino cross linker 3,3′-dithio-bis(propionate N-hydroxy succinimide ester), (DTSP, Sigma-Aldrich), which also contains a biocleavable, disulfide linkage. Alternatively, by coupling PQ to DTSP, a disulfide linkage is added which is reduced with dithiothreitol to provide a sulfhydryl group on the PQ. Alternatively, the amino group on primaquine is thiolated using 2-iminothiolane to provide thiolated chloroquine for disulfide coupling to any suitable thiolated protein or peptide CCA or carrier substance.

However, in this example, the DTSP is used to cross link PQ to an amino-containing antibody to produce a new composition.

To a solution of about 0.25 gm (1 mmole) of primaquine in 12.5 mL of about 60% DMF and 12% DMSO in water, was added about 0.35 gm (0.9 mmoles) of DTSP in 6 mL of about 16% CH₂Cl₂ in DMF. The solution of PQ-DTSP was mixed and put in the dark at rt for about 3 hours before preparing biocleavable conjugates.

PQ-Antibody.

A preferred embodiment is a biocleavable primaquine-coupled antibody substance. Any suitable antibody with available amino groups is suitably combined with a solution of PQ-DTSP to produce PQ-DTSP-antibody. The resulting PQ-antibody then contains a biocleavable disulfide linkage between the PQ and the antibody. Also, by incorporating multiple amino groups into said antibody, several PQ moieties are coupled to said antibody.

To about 1 mg of antibody, in suitable solvent, is added about 3× molar excess of PQ-DTSP solution, mixed and left at rt in the dark for 24-48 hours. The resulting product, PQ-DTSP-antibody conjugate is purified by precipitation or by Sephadex™ G25 gel exclusion chromatography and the leading fractions collected, pooled and concentrated by precipitation and/or filtration.

PQ-Insulin.

A preferred embodiment is a biocleavable primaquine-coupled insulin. Any suitable insulin with available amino groups is suitably combined with a solution of PQ-DTSP to produce PQ-DTSP-insulin. The resulting PQ-insulin then contains a biocleavable disulfide linkage between the PQ and the insulin.

To about 1 mg of insulin, in suitable solvent, is added about 3× molar excess of PQ-DTSP solution, mixed and left at rt in the dark for 24-48 hours. The resulting product, PQ-DTSP-insulin conjugate is purified by precipitation or by Sephadex™ G25 gel exclusion chromatography and the leading fractions collected, pooled and concentrated by precipitation and/or filtration.

Preparation IV-B.

Biocleavable Primaquine-Coupled Protein or Peptide Active Agents.

A thiolated chloroquine substance is coupled to any suitable protein or peptide active agent using the hetero-bifunctional crosslinking agent MAL-Fmoc-OSu, (9-hydroxymethyl-2-(amino-3-maleimidopropionate)-fluorene-N-hydroxysuccinimide). MAL-Fmoc-OSu is prepared by the methods disclosed by F. Albericio, et al, Synth. Commun. 31, 225-232 (2001) and Y. Shechter, et al, Bioconjugate Chem. 16, 913-920 (2005).

An amine-containing chloroquine substance, (i.e. primaquine (PQ), or hydroxychloroquine amine, disclosed herein) is thiolated by reacting it with 2-iminothiolane to provide a thiolated chloroquine substance.

Chloroquine-Fmoc-Insulin.

To a stirred solution of Zn²⁺ free insulin (1 micromole in 1.0 mL of 0.1 M phosphate buffer at pH 7.2) is added 1 micromole of MAL-Fmoc-OSu (about 0.050 mL of a fresh solution of MAL-Fmoc-OSu in DMF, 10 mg/mL). The reaction is carried out for 20 min at 25° C. to produce MAL-Fmoc-OSu-Insulin.

About 0.37 mL of thiolated chloroquine substance (i.e. thiolated primaquine or thiolated hydroxychloroquine) is then added to a final concentration of about 0.42 mM (0.6 mol/mol of insulin). The reaction is carried out for 2 h, and the mixture is then dialyzed overnight against water at 4-10° C. Chloroquine-Fmoc-insulin is purified from unreacted insulin and from insulin-Fmoc-MAL that had not reacted with chloroquine by preparative reverse phase HPLC chromatography (RP4 column, Hesperia Calif.), using a gradient of 20-100% mobile phase B vs water where the mobile phase B is 0.1% trifluoroacetic acid in 75% acetonitrile in water. The fractions corresponding to chloroquine-Fmoc-insulin are collected and lyophilized.

Chloroquine-HSA-Fmoc-Insulin.

Alternatively, the chloroquine substance is coupled to a protein carrier such as human serum albumin (HSA), that is also coupled to insulin through the MAL-Fmoc-OSu linkage. In this embodiment, activated chloroquine substance (i.e. HQ-aldehyde or NHS activated chloroquine, disclosed herein) is coupled to one or more amino functional groups on the HSA to give HSA-chloroquine. When the HSA-chloroquine is combined with the MAL-Fmoc-OSu-insulin (supra), the HSA-chloroquine is coupled to the MAL-Fmoc-OSu through a thiol group on the HSA to give Chloroquine-Fmoc-Insulin. The HSA thiol is exposed by treatment with 1 equivalent of dithiothreitol for 20 minutes at pH 6 (Shechter, supra).

Preparation V.

Hydroxychloroquine-Coupled Insulin.

In this example, HQ-aldehyde is directly coupled to insulin. Any suitable insulin with an available amino group is suitably combined with a solution of hydroxychloroquine-aldehyde (HQ-Aid) to produce HQ-insulin substance.

To 0.1 mL of an aqueous solution of insulin (10 micrograms) is added a 3× molar excess of 12.5% HQ-Ald in 0.2 mL water, then 0.01 mL of 0.02 M NaCO₃ to give about pH 7.5. The mixture is left at rt in the dark overnight. The Schiff s base couplings in the mixture are reduced by the addition of about 0.05 mL of 20 mM NaBH₄ solution.

The hydroxychloroquine-coupled insulin product (HQ-insulin) is purified by Superdex™ gel exclusion chromatography in buffered water. The leading fractions contained HQ-coupled insulin are determined by the presence of both HQ fluorescence (excitation 485 nm; emission 528 nm), and protein absorbance (280 nm) in the same elution peak ahead of either protein or HQ alone.

Preparation VI.

Hydroxychloroquine-Coupled Antibody.

In this example, HQ-aldehyde is directly coupled to antibody. Any suitable antibody with an available amino group is suitably combined with a solution of hydroxychloroquine-aldehyde (HQ-Ald) to produce HQ-antibody substance. Also, by derivatizing the antibody with hydrazine groups and coupling to HQ-Ald, the resulting HQ-antibody will then contain a biocleavable hydrazone linkage between the HQ and the antibody.

To 0.1 mL of an aqueous solution of antibody (10 micrograms) is added about 0.16 mL of 12.5% HQ-Ald in water, then 0.01 mL of 0.02 M NaCO₃ to give about pH 7.5. The mixture is left at rt in the dark overnight. The Schiffs base couplings in the mixture are reduced by the addition of about 0.05 mL of 20 mM NaBH₄ solution.

The hydroxychloroquine-coupled antibody product (HQ-antibody) is purified by Superdex™ gel exclusion chromatography in buffered water. The leading fractions contained HQ-coupled antibody are determined by the presence of both HQ fluorescence (excitation 485 nm; emission 528 nm), and protein absorbance (280 nm) in the same elution peak ahead of either protein or HQ alone.

Preparation VII.

Primaquine Dextran Aldehyde Conjugates.

In this example, dextran is derivatized using glycidol and oxidation to provide aldehyde groups for coupling to primaquine and other moieties.

A. Dextran-Aldehyde. To 1 mL of 15% dextran, average mw 40,000 Daltons (40 kDa) (Sigma-Aldrich), is added 0.1 mL of 1 M NaCO₃ to give a pH of about 12. To this solution is added about 0.012 mL of glycidol (40× molar), then put in the dark at rt for several days. The resulting dextran-glycidol preparation is oxidized by adding 0.05 gm of NaIO₄ and put in the dark at rt for about 2 hours. The resulting dextran-aldehyde is collected by precipitation with about 5 volumes of 100% isopropanol, cooling to −20° C. and centrifugation. The dextran-aldehyde precipitate is dissolved in water. Alternatively, it can be further purified by Sephadex™ G50 size exclusion gel chromatography in water. Aldehyde concentration is determined using HSD as described previously.

In another preferred embodiment, dextran or inulin, or other suitable polysaccharides are suitably oxidized by this method without first coupling with glycidol. The resulting aldehyde containing polysaccharide can suitably be used in place of oxidized dextran in B, C or D, below.

B. Primaquine-Dextran. Primaquine is coupled to the dextran-aldehyde by adding about a two fold (2×) molar excess of primaquine to the dextran-aldehyde in water and put in the dark for several hours at rt. The resulting primaquine-dextran conjugate is purified by Sephadex™ G50 size exclusion gel chromatography in water.

C. Primaquine-Dextran-Polypeptide Conjugate. Any suitable protein or peptide active agent containing available primary amines, is coupled to the remaining aldehydes on the primaquine-dextran-aldehyde by adding about a two or three fold molar excess of the protein or peptide (i.e. 1.6 micromoles in 0.32 mL water), to about 0.8 micromoles of primaquine-dextran-aldehyde in 0.5 mL water and about 0.040 mL of 0.02 M NaCO₃ for pH 8-9. The solution is mixed and put in the dark for several hours at rt. The resulting primaquine-dextran-polypeptide conjugate is purified by Sephadex™ G50 size exclusion gel chromatography in water.

Dextran concentration is measured as carbohydrate by a colorimetric test described previously. Poly arginine concentration is measured as amine by a colorimetric test for amines as described previously. Primaquine concentration is determined by fluorescence as described previously. Alternatively, inulin is substituted for dextran to produce inulin-aldehyde.

Preparation VIII.

Primaquine Cyclodextrin-Aldehyde Conjugates.

In this example, a cyclodextrin (CD), containing aldehyde functional groups is first prepared. The CD-aldehyde is from CD monomers, dimers, trimers or polymers previously coupled with glycidol (i.e. molar excess in water) as described herein.

A. CD-Aldehyde. To a glycidol coupled CD preparation in water (4% CD), sodium m-periodate (NaIO₄) was added directly while mixing at room temperature (rt.). The molar ratio of NaIO₄ to cyclodextrin was about 6:1, to oxidize the diols introduced with the glycidol and some of the secondary C₂-C₃ diols on the CD molecules. This produces multiple aldehydes per CD molecule. The reaction is continued at 30° C. in the dark for 6 hours to overnight. The resulting polyaldehyde CD preparation was purified by gel exclusion chromatography (G50 Sephadex™) in water, and concentrated by evaporation.

B. Primaquine-CD. Primaquine is coupled to the CD-aldehyde by adding about a two fold (2×) molar excess of primaquine to the CD-aldehyde in water and put in the dark for several hours at rt. The resulting primaquine-CD conjugate is purified by Sephadex™ G50 size exclusion gel chromatography in water or suitable MetOH/water solvent.

C. Primaquine-CD-Polypeptide Conjugate. Any suitable protein or peptide active agent containing available primary amines, is coupled to the remaining aldehydes on the primaquine-CD-aldehyde by adding about a two or three fold molar excess of polypeptide to the primaquine-CD-aldehyde in water and put in the dark for several hours at rt. The resulting primaquine-CD-poly arginine conjugate is purified by Sephadex™ G50 size exclusion gel chromatography in water.

Alternatively, the CD aldehyde preparation in this example is alpha, beta, or gamma cyclodextrin monomers, or dimers, trimers or polymers thereof, which have been suitably oxidized without pre-coupling to glycidol, to produce dialdehydes on the CD molecules. Also, other carbohydrates such as dextrans or inulins are oxidized to provide aldehydes with or without pre-coupling to glycidol. Cyclodextrin content is determined as carbohydrate as described previously.

Preparation IX.

Primaquine Lipid Conjugates and Micelles.

In this example, primaquine is coupled to oleic acid by two different coupling methods. To each of two tubes (A and B), containing about 0.03 micromoles of primaquine (Sigma-Aldrich) is added about 1 mL of DMF, or other suitable solvent to dissolve.

A. To primaquine solution A, is added about 0.5 mL of 1:5 CH₂Cl₂: DMF containing about 0.045 micromoles of oleic anhydride (Sigma-Aldrich), vortexed and put in the dark at rt for about 24 hours to allow coupling of the oleic anhydride to the amino groups.

B. To primaquine solution B, is added about 0.05 mL of DMF containing about 0.045 micromoles of oleic acid N-hydroxysuccinimide ester (Sigma-Aldrich), vortexed and put in the dark at rt for about 24 hours to allow coupling of the oleic acid N-hydroxysuccinimide ester to the amino groups.

Both preparations A and B are quenched with about 0.005 mL of ethanolamine, vortexed and put in the dark at rt for about 24 hours. The resulting primaquine-oleic acid conjugates are purified by chromatography on C₁₈ columns using gradient elution of 10-100% acetonitrile in water. Primaquine concentration is determined by fluorescence using least squares calculation from a primaquine standard curve, as described herein. Preparations are stored at −20° C.

Under suitable conditions, hydroxychloroquine-hydrazine is substituted for primaquine. Also, other suitable N-hydroxysuccinimide esters of lipids (i.e. stearylamine), can be substituted for oleic acid. These preparations are bound to HSA carrier or, are incorporated into any suitable micelle formulation which includes a protein or peptide active agent and other amphiphilic molecules as disclosed herein to provide a micelle carrier composition.

Preparation X-A.

Biocleavable Primaquine-Gamma Globulin Conjugate.

In this example (N45B), primaquine is coupled to gamma globulin protein to provide a biocleavable primaquine protein carrier. Protein or peptide CCA or other antibody can then be coupled to the gamma globulin.

To a solution of about 0.25 gm (1 mmole) of primaquine in 12.5 mL of about 60% DMF and 12% DMSO in water, was added about 0.35 gm (0.9 mmoles) of DTSP in 6 mL of about 16% CH₂Cl₂ in DMF. The solution of PQ-DTSP was mixed and put in the dark at rt for about 3 hours before preparing a biocleavable conjugate with the gamma globulin.

To about 0.2 mg of human gamma globulin (Sigma-Aldrich) in about 0.8 mL of 0.002 M NaCO₃, pH 8, is added about 3 ml of PQ-DTSP solution (about 0.25 mmoles), mixed and left at rt in the dark for 24-48 hours. The resulting product, PQ-DTSP-gamma globulin conjugate is purified by Sephadex™ G15 gel exclusion chromatography and the leading fractions collected, pooled and concentrated. Primaquine concentration is determined by fluorescence vs. protein concentration determined by amino assay as described previously.

Also, other proteins including any antibody substances disclosed herein, can be substituted for the gamma globulin in this invention. Preferred antibody substances include antibody drug conjugates including but not limited to rituximab, trastuzumab, immunotoxins, and those disclosed or referenced herein, including references therein.

Alternatively, a carboxylated CCA (i.e. NRTI, PI, MTX) or a chloroquine substance that has been converted to an active ester (i.e. 3-nitrophenyl, N-hydroxysuccinimidyl or S-ethyl activated ester) as disclosed herein, is added to the protein or antibody in suitable buffer to couple to available amine groups.

Alternatively, hydroxychloroquine-aldehyde or primaquine-aldehyde is coupled to the antibody through available amino groups on the protein.

Oxidized Gamma Globulin. In another preferred embodiment, the carbohydrate moiety of the gamma globulin, is suitably oxidized to provide aldehydes using either NaIO₄ (A. Murayama, et al, Immunochem. 15, 532, 1978), or a suitable oxidizing enzyme such as glucose oxidase. Then, primaquine or suitably, hydroxychloroquine-hydrazine is coupled to the aldehydes on the protein to provide a biocleavable hydrazone linkage.

For instance, to about 3 mg of gamma globulin in 3 mL of PBS, pH 6.2, is added about a 50× molar excess of NaIO₄ and mixed. After reacting for about 1 hour at 4° C., the reaction is quenched with about 30× molar excess of ethylene glycol. The oxidized globulin is collected by ultra filtration (50 kDa MWCO) and reconstituted in PBS.

To the oxidized globulin is added a 20× molar excess of primaquine in suitable solvent and allowed to couple for 2-3 hours in the dark at rt. The resulting PQ-Globulin is purified by Sephadex™ gel chromatography. Alternatively, this procedure, with suitable modifications, is used to produce oxidized antibody. Also, other glycoproteins including any antibody substances disclosed herein, can be substituted for the gamma globulin.

Preparation X-B.

Biocleavable Primaquine-HSA Conjugate.

In this example, primaquine is coupled to human serum albumin (HSA) protein to provide a biocleavable primaquine protein carrier. Protein or peptide CCA or antibody is then coupled to the HSA.

To a solution of about 0.25 gm (1 mmole) of primaquine in 12.5 mL of about 60% DMF and 12% DMSO in water, was added about 0.35 gm (0.9 mmoles) of DTSP in 6 mL of about 16% CH₂Cl₂ in DMF. The solution of PQ-DTSP was mixed and put in the dark at rt for about 3 hours before preparing a biocleavable conjugate with the HSA.

To about 0.2 mg of HSA (Sigma-Aldrich) in about 0.8 mL of 0.002 M NaCO₃, pH 8, is added about 3 ml of PQ-DTSP solution (about 0.25 mmoles), mixed and left at rt in the dark for 24-48 hours. The resulting product, PQ-DTSP-HSA conjugate is purified by Sephadex™ G15 gel exclusion chromatography and the leading fractions collected, pooled and concentrated. Primaquine concentration is determined by fluorescence vs. protein concentration determined by amino assay as described previously.

Alternatively, a carboxylated protein or peptide CCA or chloroquine substance that has been converted to an active ester (i.e. 3-nitrophenyl, N-hydroxysuccinimidyl or S-ethyl activated ester) as disclosed herein, is added to the protein carrier in suitable buffer to couple to available amine groups. Alternatively, hydroxychloroquine-aldehyde or primaquine-aldehyde is coupled to the protein through available amino groups on the protein.

Preparation XI.

Biocleavable Primaquine-Peptide Conjugate.

In this example (N45), primaquine is coupled to a polylysine peptide to provide a primaquine-peptide carrier. To a solution of about 0.25 gm (1 mmole) of primaquine in 12.5 mL of about 60% DMF and 12% DMSO in water, was added about 0.35 gm (0.9 mmoles) of DTSP in 6 mL of about 16% CH₂Cl₂ in DMF. The solution of PQ-DTSP was mixed and put in the dark at rt for about 3 hours before preparing a biocleavable conjugate with the gamma globulin.

To a solution of polylysine (1 millimole) in about 10 mL of suitable solvent and/or aqueous buffer (0.002 M NaCO₃, pH 8), is added about 3 ml of PQ-DTSP solution (about 0.25 mmoles), mixed and left at rt in the dark for 2448 hours. The resulting product, PQ-DTSP-peptide conjugate is purified by Sephadex™ G15 gel exclusion chromatography and the leading fractions collected, pooled and concentrated. Primaquine concentration is determined by fluorescence vs. peptide concentration determined by amino assay as described previously.

Alternatively, a carboxylated chloroquine substance that has been converted to an active ester (i.e. 3-nitrophenyl, N-hydroxysuccinimidyl or S-ethyl activated ester) as disclosed herein, is added to the peptide in suitable buffer to couple to available amine groups.

Alternatively, hydroxychloroquine-aldehyde or primaquine-aldehyde is coupled to the peptide through available amino groups. Also, any suitable peptide, such as those containing lysine or arginine, with one or more available amino groups, is substituted for the peptide in this example. Preferably, nucleic acid (i.e. DTSP-coupled ODN) can also be coupled to the peptide through biocleavable linkages.

Preparation XII.

Primaquine-PEG Conjugate.

In this example, primaquine is coupled to a diepoxy PEG to provide a primaquine-PEG (PQ-PEG) conjugate. The conjugate is then thiolated to provide sulfhydryl groups for coupling other moieties.

A. Primaquine-PEG. To about 0.03 micromoles of primaquine (Sigma-Aldrich) in about 10 mL of DMF is added about 700 micrograms (0.03 micromoles) of polyethylene glycol diglycidyl ether, “PEG-DE”, mw about 23,250 (Sigma-Aldrich #47,569-6). The solution is mixed and put in the dark at rt for 34 days.

Remaining epoxy groups are quenched by adding 30 micrograms (0.12 micromoles) of sodium thiosulfate in 0.010 mL water, mixed and kept at rt in the dark for 2 days. To this solution is added about 0.23 milligrams of dithiothreitol (DTT) in about 1 mL of water, mixed and kept at rt in the dark for about 3 hours to reduce coupled sodium thiosulfate to sulfhydryl groups on the PQ-PEG conjugate.

B. Purification. The preparation is fractionated by size exclusion gel chromatography on a Sephadex™ G25 column in suitable solvent (i.e. 10% MetOH/water) as the mobile phase. Fractions are collected and monitored for primaquine fluorescence as described previously. The leading fractions that contain PEG with primaquine fluorescence indicate that PQ is coupled to the PEG. The PQ-PEG fractions are pooled and concentrated by evaporation in the dark, under flowing nitrogen.

Alternatively, the PEG-DE is first coupled to hydrazine through the epoxy groups to produce PEG-hydrazine. Then hydroxychloroquine-aldehyde or primaquine-aldehyde is coupled to the hydrazine on the PEG to provide acid labile linkages as described previously. Alternatively, any suitable diamino or polyamino compound is used in place of hydrazine (i.e. PEG-dilysine), and/or primaquine or hydroxychloroquine-amine is coupled to the PEG-hydrazine through suitable cross linkers.

Alternatively, a carboxylated protein or peptide CCA or chloroquine substance that has been converted to an active ester (i.e. 3-nitrophenyl, N-hydroxysuccinimidyl or S-ethyl activated ester) as disclosed herein, is added to the PEG-hydrazine or PEG-dilysine in suitable buffer to couple to available amine groups.

Alternatively, the PEG-DE is first coupled to sodium thiosulfate through the epoxy groups, then reduced with DTT to produce sulfhydryl-PEG. Then sulfhydryl derivatized (thiolated) primaquine or sulfhydryl derivatized (thiolated) hydroxychloroquine is coupled to the sulfhydryl groups on the PEG to provide biocleavable disulfide linkages as described previously.

Preparation XIII.

Pendant PEG-Hydrazine For Biocleavable Linkages.

In this example, pendant polyethylene glycol (SunBio USA, mw 20 KDa) with approximately 15 propionic acid side chains (PaPEG) is coupled to hydrazine through available carbonyl groups on the PEG. This provides side chains with terminal hydrazine moieties. The hydrazine groups can then be coupled to moieties containing aldehyde groups to provide biocleavable, acid-labile hydrazone linkages.

A. PaPEG-Hydrazine. Into about 20 ml of water, about 5 gm of pendant PEG was dissolved, the pH was about 5. Based on the manufacturer's value of 15 moles of propionic acid per mole of PaPEG, there was about 0.375 mmoles of carboxylic acid present. In a separate container, 1.8 ml of hydrazine hydrate (64%, fw 50.06) was neutralized to pH 7 with about 6.25 ml of 5N HCl, to give a final concentration of about 0.225 ml hydrazine per ml of solution.

A thirty-fold molar excess (30×) of hydrazine (4 ml of hydrazine solution) was added to the PaPEG solution and mixed with a magnetic stirrer. After about 2 minutes, a twenty-fold molar excess (20×=1.45 gm) of N-(3-Dimethylaminopropyl)-N′-Ethylcarbodiimide (EDC, fw 191.7), was added to the solution of PaPEG and mixed thoroughly. The pH was about 6. The solution was allowed to react overnight at room temperature (rt).

B. Purification. The reaction mixture was fractionated on a Sephadex™ G25 column equilibrated and eluted with 0.005 M HCl in water. The fractions are analyzed for refractive index. They are also analyzed for primary amine using a colorimetric test described previously. The leading fractions with corresponding high refractive index and amine content are pooled and concentrated by evaporation under nitrogen gas. The resulting product (PaPEG-Hzn), is PaPEG with hydrazine functional groups covalently coupled to the propionic acid moieties.

The PaPEG-Hzn can now have any suitable moiety with a terminal aldehyde group coupled to the available hydrazine groups. This will provide an acid labile hydrazone linkage described herein. Alternatively, any suitable diamino compound is used in place of hydrazine.

Alternatively, any suitable chloroquine substance, intercalator, or other moiety with a terminal active ester is coupled to the amine as described herein. Alternatively, the hydrazine (or amino) groups are thiolated using SPDP or 2-iminothiolane as described herein to provide thiols for disulfide coupling to a suitable thiolated protein or peptide active agent.

Alternatively, using coupling agents described herein, the terminal hydrazine groups are coupled to a diamino, Fmoc half-protected biocleavable peptide containing any suitable biocleavable linkage such as GFLG, Phe-Leu, Leu-Phe or Phe-Phe, among others. The Fmoc groups are then removed to provide unprotected amino groups for subsequent coupling to an intercalator. Alternatively, said biocleavable peptide can include a sulfhydryl group at one end for subsequent coupling to a thiolated antibody (i.e. disulfide coupling), or amino-derivatized antibody using a bifunctional cross linking agent.

Alternatively, a carboxylated CCA (i.e. NRTI, PI, MTX) or chloroquine substance that has been converted to an active ester (i.e. 3-nitrophenyl, N-hydroxysuccinimidyl or S-ethyl activated ester) as disclosed herein, is added to the PaPEG-hydrazine in suitable buffer to couple to available amine groups.

Alternatively, the hydroxyl end groups on the PEG backbone are suitably derivatized and coupled to suitable targeting molecules, transduction vectors, or grafted polymers using other coupling groups such as succinimide, N-succinimidyl, bromoacetyl, maleimide, N-maleimidyl, oxirane, p-nitrophenyl ester, or imidoester. Alternatively, aldehydes coupled to hydrazine to give amino-aldehyde (Schiff s base) bonds are reduced with NaBH₄ to stabilize them.

Preparation XIV.

Maleimido or Iodo Carrier Substances Coupled to a Thiolated Moiety.

In this example, an amino-containing carrier substance is derivatized to contain a maleimide or an iodo reactive group. Then a chloroquine substance or protein or peptide active agent, intercalator, targeting molecule, transduction vector or other moiety is suitably thiolated as described herein before coupling it to the derivatized carrier substance. There are well known methods for derivatizing the primary amine on the carrier substance (i.e. protein, PEG) to provide a maleimido group. For instance, a bifunctional (succinimidyl-maleimido) cross linker described herein, such as MBS or SMPB is coupled to the primary amine to provide free maleimide groups. Upon reaction with a thiolated moiety, a stable thioether bond is formed.

Alternatively, iodo-carrier substances such as iodo-polyethylene glycol (Iodo-PEG) carriers are prepared for coupling to a sulfhydryl group on a chloroquine substance or protein or peptide active agent, intercalator, targeting molecule, transduction vector or other moiety. For instance, NHS esters of iodoacids are coupled to the amino-containing carrier substances. Suitable iodoacids for use in this invention are iodopropionic acid, iodobutyric acid, iodohexanoic acid, iodohippuric acid, 3-iodotyrosine, among others. Before coupling to the amino-carrier substance, the appropriate Iodo-NHS ester is prepared by known methods. For instance, equimolar amounts of iodopropionic acid and N-hydroxysuccinimide are mixed, with suitable carbodiimide, in anhydrous dioxane at RT for 1-2 Hrs, the precipitate removed by filtration, and the NHS iodopropionic acid ester is collected in the filtrate. The NHS iodopropionic acid ester is then coupled to the amino-carrier substance.

Preparation XV.

Amphiphilic Cyclodextrin.

In this example, a mixture of amphiphilic cyclodextrin dimers, trimers and polymers with alkyl carbon chains attached is prepared for use as carrier substances. The cyclodextrins are cross-linked through hydroxyl groups using 1,4 butanediol diglycidyl ether (BDDE). Excess BDE molecules coupled at one end to the CD provide terminal oxirane groups that are subsequently thiolated by reaction with thiosulfate and reduction. Alkyl carbon chains are coupled to the CD derivatives using a “long chain epoxy” that couples to other available hydroxyl groups (CD8B).

A. Cross-linking with BDDE. Into 125 ml of hot water (70-80° C.) adjusted to pH 4.5-5 with 0.05 ml 6 N HCl, is dissolved 2.84 gm of beta cyclodextrin (0.0025 moles). To this solution 4.1 ml of BODE (about 0.0125 moles) is added with mixing and heating for about 2 hours.

B. Coupling with a Long Chain Epoxy. The mixture is adjusted to pH >10 with 1 M KOH and 1.28 gm (about 0.005 moles) of dodecyl/tetradecyl glycidyl ether (DTGE) is added and mixed vigorously. The solution is periodically mixed for about 1.5 hours, heated for about 3 hours and then left at room temperature (rt) overnight. The resulting solution is light yellow and turbid.

C. Thiolation with Na Thiosulfate. To the reheated mixture, 6 gm (about 0.025 moles) of sodium thiosulfate is added and mixed. After about 1 hour, the pH is adjusted to 7 with KOH and the solution was heated for about 3.5 hours more. Excess DTGE was removed by chilling to solidify the DTGE and the solution was decanted. The mixture was dialyzed against a continuous flow of distilled water in 500 molecular weight cutoff (mwco) tubing (Spectra/Por CE) for about 40 hours. The solution was concentrated by evaporation to 8 ml to give a clear, light yellow solution.

To the mixture, 8 ml of water and 0.96 gm (about 0.0062 moles) of dithiothreitol (DTT) was added, mixed and left overnight. The turbid solution was then dialyzed against a continuous flow of distilled water in 500 mwco tubing (Spectra/Por CE) for about 40 hours. The solution was concentrated by evaporation to 3.7 ml to give a clear, yellow solution. Total yield based on dry weight was 2.276 gm.

D. Column Chromatography. The mixture was fractionated on a Sephadex™ G15 column (2.5×47 cm) in water. The fractions are tested for relative carbohydrate and thiol concentration as described previously. Fractions with corresponding peak concentrations of carbohydrate and thiol are pooled and concentrated by evaporation. The final volume was 2.2 ml and the total yield based on dry weight was 1.144 gm. The resulting amphiphilic CD polymer was highly water soluble and amorphous (glassy) when dried.

E. Coupling With Thiolated Moieties. The amino groups on moieties such as amino-derivatized chloroquine substances (i.e. primaquine), or trioxsalen amine and other amino-moieties are thiolated using SPDP or 2-iminothiolane as described previously. The thiolated moieties are then coupled to the carrier substance through disulfide linkages using thiol-disulfide interchange as described previously. Alternatively, other thiolated moieties such as targeting molecules, transduction vectors and grafted polymers are coupled through disulfide linkages.

Alternatively, to produce other suitable hydrophobic CD derivatives, other alkyl chains are introduced by substituting suitable alkyl epoxy compounds for the one used in this example. For instance 1,2-epoxy derivatives of any suitable alkane such as propane, butane, pentane, hexane, octane, decane and dodecane are substituted. Other useful epoxies such as glycidyl isopropyl ether, glycidyl methacrylate and glycidyl tosylate can be substituted. Also certain aromatic epoxies or heterocyclic epoxies can be substituted such as benzyl glycidyl ether, (2,3-epoxypropyl) benzene, 1,2-epoxy-3-phenoxypropane, exo-2,3-epoxynorborane, among others.

Alternatively, the CD polymer is suitably derivatized with other coupling groups such as succinimide, N-succinimidyl, bromoacetyl, maleimide, N-maleimidyl, oxirane, p-nitrophenyl ester, or imidoester. Alternatively, the CD polymer is coupled to a polypeptide containing any suitable biocleavable linkage such as Phe-Leu, Leu-Phe or Phe-Phe, among others. Or, the CD polymer is suitably derivatized to provide a CD-block with an N carboxyanhydride for subsequent copolymerization into PEO-block copolymers.

Combinations for this invention can include the covalent coupling of an alpha CD with a beta CD, an alpha CD with a gamma CD, a beta CD with a gamma CD and polymers with various ratios of alpha, beta and gamma cyclodextrins.

Preparation XVI.

Carriers From Hydroxylated Polymers.

These are methods for synthesizing antibody carrier compositions to provide for coupling to any suitable protein or peptide, targeting molecule, transduction vector, or other moiety with a suitable functional group. The targeting molecule is a suitable protein, including antibody substances, lectins, avidins and streptavidin or ligands.

A. Preparation of NHS-Carrier Substances. A carrier substance with available hydroxyl groups such as carbohydrates (i.e. CD, or inulin), PEG and other grafted polymers described herein, is derivatized to provide an NHS ester. In a suitable anhydrous solvent such as DMF, the carrier substance is coupled to acetic anhydride and purified as described herein, to provide carboxyl groups. Then, the carboxylic acid group is reacted with N-hydroxysuccinimide and an aromatic carbodiimide such as N,N-dicyclohexyl carbodiimide, at approximately equimolar ratios and reacted at rt for 1-3 Hrs. The product, N-hydroxysuccinimide carrier (i.e. NHS-PEG), is separated in the filtrate from precipitated dicyclohexylurea, collected by evaporation and purified by chromatography.

Under appropriate conditions, NHS-carrier substances are prepared by coupling NHS esters directly to an amino derivatized carrier substance. Preferably, the NHS ester is a bifunctional NHS coupling agent with a suitable spacer. Suitable NHS coupling agents for use in this invention have been previously described, including DSS, bis(sulfosuccinimidyl)suberate (BS³), DSP, DTSSP, SPDP, BSOCOES, DSAH, DST, and EGS, among others.

The NHS-carrier substance can now be coupled to any suitable amino-containing chloroquine substance, protein or peptide active agent, targeting molecule, transduction vector, or other amino-containing moiety using methods for coupling active esters described herein.

B. Thiolated Carrier Substances. Alternatively, thiolated carrier substances are prepared from amino-containing carrier substances as described herein. Then, through disulfide coupling, the carrier substance is coupled to other available sulfhydryls on the desired thiolated intercalator, targeting molecule, transduction vector, or other moiety. Alternatively, a sulfhydryl-containing carrier substance (i.e. thiolated PEG) is coupled to any maleimide derivative of a transduction vector, targeting molecule, or biotin, (e.g. biotin-maleimide) or iodoacetyl derivatives such as N-iodoacetyl-N′-biotinylhexylenediamine.

C. Maleimido or Iodo-Carrier Substances. Alternatively, maleimide or iodo derivatized carrier substances, are prepared from amino-containing carrier substances of this invention using well known methods. Such carrier substances are suitable for coupling to native or introduced sulfhydryls on the desired chloroquine substance, protein or peptide active agent, intercalator, targeting molecule, transduction vector, or other moiety.

A maleimido group is added to an amino-carrier substance suitably prepared as described previously, by coupling a suitable hetero-bifunctional coupling agent to the available amino group. The hetero-bifunctional coupling agent consists of a suitable spacer with a maleimide group at one end and an NHS ester at the other end. Examples are previously described and include MBS, SMCC, SMPB, among others. The reaction is carried out so that the NHS ester couples to the available amino group on the carrier substance, leaving the maleimide group free for subsequent coupling to an available sulfhydryl on an intercalator, transduction vector, targeting molecule, or other moiety.

Under appropriate conditions, iodo-carrier substances (i.e. Iodo-PEG) can also be prepared for coupling to sulfhydryl groups. For instance, NHS esters of iodoacids are coupled to the amino-carrier substances as described previously.

Preparation XVII.

Biotinylated Carriers.

Carrier substances defined herein are coupled to biotin by a variety of known biotinylation methods suitably modified for use with the carrier substances of this invention. For instance, an amino-containing carrier substance is combined with an active ester derivative of biotin in appropriate buffer such as 0.1 M phosphate, pH 8.0, reacting for up to 1 hour at room temperature. Examples of biotin derivatives that are useful are, biotin-N-hydroxysuccinimide, biotinamidocaproate N-hydroxysuccinimide ester or sulfosuccinimidyl 2-(biotinamino)ethyl-1,3′-dithiopropionate, among others.

Through the use of suitable protection and deprotection schemes, as needed, any carrier substance of the instant invention are coupled to biotin or a suitable derivative thereof, through any suitable coupling group. For instance, biocytin is coupled through an available amino group to any active ester derivatized carrier substance described herein.

The resulting biotinylated carrier substance is then coupled to any suitable avidin or streptavidin that contains the desired chloroquine substance, protein or peptide active agent, or intercalator. The avidin or streptavidin may also contain a targeting molecule, transduction vector or other moiety.

Preparation XVIII.

Avidin Carriers.

Avidin or streptavidin carrier substances defined herein are coupled to biotinylated moieties including biotinylated chloroquine substances, biotinylated protein or peptide active agents and other moieties. Biotinylated moieties can also include targeting molecules or transduction vectors. For instance, streptavidin is suitably carboxylated without impairing the biotin binding sites. The carboxyl groups are then derivatized to provide one or more active esters as described herein. Primaquine, quinacrine amine, mefloquine amine or hydroxychloroquine amine is then coupled to the activated esters as described herein. Biotinylated moieties are coupled to the streptavidin carrier substance before or after other moieties are coupled to the active esters. Alternatively, moieties such as targeting molecules, intercalators or transduction vectors are coupled to the active esters through their amino groups.

Preparation XIX.

Amino Acid-Coupled Chloroquine-Substance.

The purpose of these compositions is to deliver the chloroquine or other chloroquine substance at the same site as its peptide or protein active agent, thereby reducing the overall dosage needed. A peptide composition has been discovered that includes the coupling of a chloroquine substance as defined herein, to any suitable transduction vector or peptide active agent of this invention. The following methods can be suitably modified for coupling amino derivatized chloroquine substances based on the disclosures of Z. Wang, et al, in JACS 117, 5438-5444 (1995) and references therein, for the preparation of amino acid-coupled chloroquine substances.

Primaquine-Coupled N-alpha-Fmoc-L-Aspartic Acid alpha-Tert-Butyl Ester.

1. Activated Ester N-alpha-Fmoc-aspartic acid alpha-tert-butyl ester. To prepare the activated aspartic acid ester, 1-hydroxybenzotriazole (HOBt) (0.5 mmoles), dissolved in about 2 mL of dry DMF is added to an ice-cooled solution of N-alpha-Fmoc-aspartic acid alpha-tert-butyl ester (0.5 mmoles) in about 2 mL of dry dichloromethane, followed by the addition of DCC (dicyclohexyl carbodiimide, 0.5 mmoles) in 2 mL of dry dichloromethane.

The reaction mixture is stirred at 0° C. for 1 h then at room temperature for 2 hours. The reaction mixture is filtered and activated ester is collected from the filtrate that is evaporated to dryness. The activated ester is redissolved in about 4 mL of dry dichloromethane.

2. Coupling to Primaquine. To form a free base, primaquine HCl salt (0.4 mmoles) in dry DMF is mixed with N,N-diisopropylethylamine (0.4 mmoles) and stirred at room temperature for 2-5 minutes. The coupling reaction is started by adding the free base of primaquine (PQ) to the activated ester solution. The final pH of the coupling reaction is adjusted to 8.0 by the addition of about 0.05 mL of diisopropylethylamine, and the mixture is stirred for about 20 minutes. The reaction mixture is concentrated to dryness under reduced pressure. The primaquine-coupled aspartic acid tert butyl ester is purified by recrystallization in suitable solvent (i.e. methanol) and dried. Alternatively, the product can be purified by column chromatography.

3. Primaquine-Coupled Fmoc-L-Aspartic Acid. To prepared primaquine-coupled Fmoc-L-aspartic acid (PQ-Fmoc-aspartate), the PQ-coupled aspartic acid tert-butyl ester (0.3 mmol.) is dissolved in dry dichloromethane or other suitable solvent and cooled to 0° C. To this solution is added about 2 mL of trifluoroacetic acid and stirring is continued at 0° C. for about 2 hours, followed by stirring at room temperature until the tert-butyl ester is removed. The reaction mixture is concentrated under reduced pressure without heating to dryness. The PQ-Fmoc-aspartate is purified by recrystallization in suitable solvent (i.e. methanol) and dried. Alternatively, the product can be purified by column chromatography.

4. Primaquine-Coupled Transduction Vector Peptide. All Fmoc-amino acids, piperidine, 4-(dimethyl-amino)pyridine, dichloromethane, DMF, HOBT, 2-(1H-benzotriazol-1-yl)-1,1,3,3-tetramethyl-uronium hexafluorophosphate (HBTU), N,N-diisopropyl ethylamine and HMP-linked polystyrene resin are available from Applied Biosystems Division, Perkin-Elmer. Trifluoroacetic acid, 1,2-ethanedithiol, phenol and thioanisol are available from Sigma.

One or more PQ-Fmoc-aspartate moieties can be incorporated into any suitable peptide including transduction vector peptides (i.e. Tat-derived from amino acids 42-72). For instance, the desired transduction vector peptide is first synthesized on an Applied Biosystems 431A peptide synthesizer using standard FastMoc protocols. Then primaquine attachment to the N-terminus of the transduction vector peptide is achieved by using PQ-Fmoc-aspartate (step 3, above) and standard FastMoc coupling reagents. Cleavage and deprotection of the peptide are carded out in 2 mL of Reagent K for 6 h at room temperature. Reagent K contains 1.75 mL of TFA, 0.10 mL of thioanisole, 0.10 mL of water and 0.05 mL of 1,2-ethanedithiol. After cleavage from the resin, the PQ-TV-peptide is purified by HPLC.

With suitable modifications in these methods, other amino-containing chloroquine substances are substituted for the primaquine HCl. Some substitution examples are primaquine diphosphate, amino-hydroxychloroquine, amino-derivatized mefloquine and amino-derivatized amodiaquine. Also, with suitable modifications in these methods, other suitable Fmoc-amino acids can be substituted for the Fmoc-aspartate.

In another preferred embodiment, any suitable chloroquine substance is thiolated (Thio-Chloroquine Substance), to provide a sulfhydryl functional group. Then, one or more said Thio-Chloroquine Substances are incorporated into any suitable peptide, including transduction vector peptides, or carrier substances that contain at least one cysteine amino acid. The Thio-Chloroquine Substance is suitably coupled to the cysteine using a disulfide exchange reaction to produce a disulfide linkage.

Preparation XX.

Biotinylated Chloroquine.

Chloroquine substances defined herein are coupled to biotin by a variety of known biotinylation methods suitably modified for use with the chloroquine substances of this invention. For instance, an amino-containing chloroquine substance (i.e. primaquine or amino-hydroxychloroquine, amino-derivatized mefloquine, amino-derivatized amodiaquine) is combined with an active ester derivative of biotin in appropriate buffer such as 0.1 M phosphate, pH 8.0, reacting for up to 1 hour at room temperature. Examples of biotin derivatives that are useful are, biotin-N-hydroxysuccinimide, biotinamidocaproate N-hydroxysuccinimide ester or sulfosuccinimidyl 2-(biotinamino)ethyl-1,3′-dithiopropionate, among others.

Through the use of suitable protection and deprotection schemes, as needed, any chloroquine substance of the instant invention are coupled to biotin or a suitable derivative thereof, through any suitable coupling group. For instance, biocytin is coupled through an available amino group to any active ester derivatized chloroquine substance (HO-Suc, MQ-Suc or HQ-aldehyde) described herein. The resulting biotinylated chloroquine substance is then noncovalently coupled to any suitable avidin or streptavidin that contains the desired protein or peptide CCA, active agent, or intercalator. The avidin or streptavidin may also contain a targeting molecule, transduction vector, quinacrine or other moiety.

Chloroquine-Coupled CCAs.

Chloroquine substances defined herein are coupled to any suitable chloroquine combinative agent (CCA) defined herein by a variety of known coupling methods including those disclosed or referenced herein, suitably modified for use with the chloroquine substances of this invention. For coupling chloroquine substances to any suitable protein or peptide CCAs herein, the derivatives and coupling methods disclosed by K Parang, et al, Curr. Med. Chem. (2000) 7, 995-1039, including references therein, can be used in this invention with suitable modification, which are hereby incorporated herein by reference. Preferred are the coupling of chloroquine substances with protein or peptide CCA derivatives that include but not limited to protein or peptide CCA carboxylic acid esters.

Preparation XXI.

Chloroquine-Coupled Protein-CCA.

A chloroquine-coupled protein-CCA composition is prepared by coupling a suitable chloroquine substance to any protein or peptide active agent described or referenced herein, such as insulin.

In this invention, said chloroquine-coupled protein-CCA composition may also include any protein carrier substance that includes but is not limited to plasma protein carrier substances, cellular protein carrier substances, protamines, noncovalent coupling proteins, any antibody substances, oxidized antibodies, oxidized glycoproteins and peptide carrier substances defined or referenced herein. Preferred protein carrier substances include, but are not limited to, humanized antibodies, synthetic antibodies, therapeutic antibodies and antibody fragments, avidins, streptavidins, any HSA, any protamines, poly arginines, transduction vectors and receptor binding peptides.

Before or after preparation, said protein-CCA also provides at least one functional group for coupling to any chloroquine substance. If desired, said functional group can be added by derivatization of said protein carrier substance using well known methods such as acylation, amination, thiolation, etc., disclosed or referenced herein.

In one example of a preferred chloroquine-coupled protein-CCA, the hydroxyl functional group of the protein or peptide CCA is esterified with a carboxylate group on a protein carrier substance (i.e. HSA or antibody fragment) in the presence of DCC and 4-(dimethylamino) pyridine (DMAP) in suitable buffer or solvent. Said coupling can also include an intermediate protein carrier substance between the protein or peptide CCA and human transferrin, with functional group available for additional coupling to a chloroquine substance.

Another preferred method is to prepare a cysteine-containing protein-CCA ester wherein a carboxylate group is available and other functional groups are suitably protected. The procedure is based on the methods of S Gunaseelan, et al, Bioconj. Chem. (2004) 15, 1322-1333 and references therein, which are incorporated herein.

The active hydroxyl functional group of any suitable protein or peptide CCA is esterified with the carboxylate group on an Fmoc and/or Trt protected protein carrier substance (i.e. transduction vector) using DIC and DMAP as coupling reagents. The protein-CCA ester is prepared for additional coupling to chloroquine substances by deprotection of the amines by Fmoc removal with piperidine and by deprotection of the sulfhydryls by Trt removal with TFA.

Alternatively, the protein or peptide CCA can be esterified by derivatizing an available hydroxyl functional group with succinic anhydride to give carboxylated CCA. The carboxylated CCA is then coupled to a suitable chloroquine-coupled protein carrier substance (i.e. protamine) in the presence of DCC and DMAP based on the methods of B M Tadayoni, et al, Bioconj. Chem. (1993) 4, 139-145.

Alternatively, the carboxylated CCA is converted to an active ester (i.e. NHS) as disclosed herein, and added to the protein in suitable buffer to couple available amine groups. In these methods, protection of certain amino groups (i.e. Fmoc) or sulfhydryls (i.e. Trt) on the protein can be done before esterification and then deprotected afterward so they are available for additional coupling, using well known methods. Examples for coupling of the chloroquine substance to the protein-CCA may also include methods described for preparing the amino acid-coupled chloroquine substances described herein. Also, any suitable protein carrier substance can be substituted in these examples.

A. In one example, HSA-insulin is combined with an equimolar or excess amount of hydroxychloroquine aldehyde (HQ-Ald), described herein, in PBS and allowed to covalently couple. The product is collected and purified by precipitation or column chromatography. The resulting product is a new aldehyde-ester composition, HQ-HSA-insulin. The chloroquine aldehyde can be substituted for any other chloroquine substance aldehydes such as PQ-aldehyde or MQ-aldehyde.

B. In another example, a streptavidin-protein active agent conjugate is combined with an equimolar or excess amount of 3-nitrophenyl, N-hydroxysuccinimidyl or S-ethyl activated ester chloroquine substance, described herein, such as hydroxychloroquine NHS ester (HQ-NHS), in suitable buffer or solvent and allowed to covalently couple. The product is collected and purified by precipitation or column chromatography. The resulting product is a new ester composition, HQ-streptavidin-protein. The HQ-NHS can be substituted for any other activated ester chloroquine substances such as PQ-NHS or MQ-NHS.

C. In another example, thiolated protamine-insulin is combined with an equimolar or excess amount of thiolated chloroquine substance, described herein, such as thiolated hydroxychloroquine amine (HQ-S), in suitable solvent or PBS and allowed to covalently couple by disulfide bonding. One preferred method is to use thiol-disulfide interchange wherein the thiolated chloroquine substance is first activated with 2DD, described herein, then combined with the thiolated protamine-insulin. The product is collected and purified by precipitation or column chromatography. The resulting product is a new disulfide-ester composition, HQ-protamine-insulin.

Also, with suitable modification, two or more protein or peptide CCAs are coupled to any protein carrier substance and include coupling to a chloroquine substance. Also, any suitable fatty acid, lipid, steroid, surfactant substance, biotin, targeting moiety or transduction vector disclosed or referenced herein can also be coupled to the protein in any chloroquine-coupled protein-CCA composition disclosed herein.

With suitable modification of these examples, protein or peptide CCAs are coupled to protein carrier substances including but not limited to glycoproteins, antibody substances and HSA, using the methods disclosed by G Molema, et al, J Med. Chem. 1991 March;34(3):113741 and J A Kamps, et al, Biochim Biophys Acta. (1996) 1278(2):183-90, and references therein, and are coupled to chloroquine substances in this invention. The resulting chloroquine-substance-protein carrier-CCA conjugates may also be incorporated into micelles.

Preparation XXII.

Chloroquine-Coupled Polymer-CCA

A chloroquine-coupled polymer-CCA composition is prepared by coupling a suitable chloroquine substance to any polymer or grafted polymer carrier substance that is also coupled to any protein or peptide CCA described or referenced herein.

In this invention, a grafted polymer substance includes but is not limited to any grafted polymers, amphiphilic grafted polymers and cationic grafted polymers defined or referenced herein. Before or after preparation, said polymer-CCA also provides at least one functional group for coupling to any chloroquine substance. If desired, said functional group can be added by coupling amino acids to the grafted polymer and/or derivatization of said grafted polymer using well known methods such as acylation, amination, thiolation, etc., disclosed or referenced herein.

One example of a preferred polymer-CCA such as pegylated insulin is readily prepared based on the methods of S K Aggarwal, et al, J Med. Chem.(1990) 33(5):1505-10. The hydroxyl function of a suitable protein or peptide CCA is esterified with a carboxylate group on the grafted polymer (i.e. APEG, PLGA) in the presence of DCC and 4-(dimethylamino) pyridine (DMAP) in suitable buffer or solvent. The polymer is suitably modified to provide amino groups for additional coupling of an NHS-ester chloroquine substance.

While the invention has been described with reference to certain specific embodiments, it is understood that changes may be made by one skilled in the art that would not thereby depart from the spirit and scope of the invention, which is limited only by the claims appended hereto.

What is claimed is:

1. A chloroquine-coupled composition comprising:
a) a chloroquine substance covalently coupled to;
b) an active agent selected from the group consisting of protein active agents and peptide active agents.

2. The composition of claim 1 wherein said chloroquine substance (a) is selected from the group consisting of quinoline compounds, 4-aminoquinoline compounds, 2-phenylquinoline compounds, chloroquines, hydroxychloroquines, amodiaquins, amopyroquines, halofantrines, mefloquines, nivaquines, primaquines, tafenoquines, quinone imines, chloroquine analogs or derivatives, (−)-enantiomers of chloroquine, (−)-enantiomers of hydroxychloroquine and amino, thio, phenyl, alkyl, vinyl and halogen derivatives thereof.

3. The composition of claim 1 wherein said active agent is selected from the group consisting of antibody substances, synthetic antibodies, polypeptide hormones, calcitonins, enkephalins, erythropoietin, EPO derivatives, follical stimulating hormone, FSH derivatives, human growth hormone, HGH derivatives, glucagons, gonadotropin-releasing hormones, human insulin and other insulins, insulin fragments, pegylated insulin and other insulin derivatives, alpha interferons, beta interferons, gamma interferons, pegylated interferons, interleukins, pegylated interleukins, laminin fragments, tumor necrosis factors, TNF, TNF alpha, TNF beta, TNFα, 4-1BBL, APRIL, BAFF, CD27L, CD30L, CD40L, FasL, LIGHT, OX40L, RANKL, TRAIL, TWEAK and VEG1, vaccine antigens, recombinant proteins, recombinant polypeptides, recombinant bioactive peptides and analogs and derivatives thereof.

4. The composition of claim 1 further comprising a targeting molecule coupled to said composition.

5. The composition of claim 1 further comprising a transduction vector coupled to said composition.

6. The composition of claim 1 wherein said covalent coupling of said chloroquine substance of (a) to active agent of (b) is through a biocleavable linkage selected from the group consisting of an acid labile linkage, a disulfide linkage, a protected disulfide linkage, an ester linkage, an ortho ester linkage, a phosphonamide linkage, a biocleavable peptide linkage, an azo linkage and an aldehyde bond.

7. The composition of claim 1 wherein said active agent is selected from the group consisting of alemtuzumab, mitumomab, epratuzumab, bevacizumab, Brevarex™, CDP860, trastuzumab, HuMax-CD20, HuMax-CD4, HuMax-EGFr, huN901-DM1, IDEC-114, IGN-101, MLN2704, bivatuzumab mertansine, MLN591 RL, gemtuzumab ozogamicin, pertuzumab, orthoclone OKT3, OvaRex, pemtumomab, Raptiva™, Reopro™, rituximab, SGN-15, SGN-30, SGN-35, SGN-75, Simulect™, Synag s™, TheraCIM hR3, Tysabri™, Vitaxin™, Xolair™, Zenapax™, RAV12, CAT-3888, CAT-8015, CAT-354, GC-1008, adalimumab, ABT-874, LymphoStat-B™, HGS-ETR1, HGS-ETR2, ABthrax™, MYO-029, MT201, IMC-11F8, IMC-1121B, Ch14.18. chimeric mAb, WX-9250, cG250 chimeric mAb, MDX-010 humanized mAb, panitumumab, human mAb, Remitogen, infliximab, LM-1, NORM-1, NORM-2, SAM-6, CM-1, CM-2, PM-1 and PM-2, including fractions and derivatives thereof.

8. A chloroquine-coupled composition comprising:
a) a chloroquine substance covalently coupled to;
b) a carrier substance and;
c) wherein said carrier substance is coupled to an active agent selected from the group consisting of protein active agents and peptide active agents.

9. The composition of claim 8 wherein said chloroquine substance (a) is selected from the group consisting of quinoline compounds, 4-aminoquinoline compounds, 2-phenylquinoline compounds, chloroquines, hydroxychloroquines, amodiaquins, amopyroquines, halofantrines, mefloquines, nivaquines, primaquines, tafenoquines, quinone imines, chloroquine analogs or derivatives, (−)-enantiomers of chloroquine, (−)-enantiomers of hydroxychloroquine and amino, thio, phenyl, alkyl, vinyl and halogen derivatives thereof.

10. The composition of claim 8 wherein said active agent is selected from the group consisting of antiviral CCA, antimicrobial CCA, anticancer CCA, antiparasitic CCA, protein or peptide CCA, immune disorder CCA, neurological CCA, toxins and abused drug CCA, and small hormonal CCA and analogs and derivatives thereof.

11. The composition of claim 8 wherein said carrier substance is selected from the group consisting of avidins, streptavidins, antibody substances, albumins, grafted polymers, micelles and dendrimers.

12. The composition of claim 8 further comprising a targeting molecule coupled to said carrier substance.

13. The composition of claim 8 further comprising a transduction vector coupled to said carrier substance.

14. The composition of claim 8 wherein said covalent coupling of said chloroquine substance of (a) to carrier substance (b); is through a biocleavable linkage selected from the group consisting of an acid labile linkage, a disulfide linkage, a protected disulfide linkage, an ester linkage, an ortho ester linkage, a phosphonamide linkage, a biocleavable peptide linkage, an azo linkage and an aldehyde bond.

15. The composition of claim 8 wherein said covalent coupling of said active agent of (c) to carrier substance (b); is through a biocleavable linkage selected from the group consisting of an acid labile linkage, a disulfide linkage, a protected disulfide linkage, an ester linkage, an ortho ester linkage, a phosphonamide linkage, a biocleavable peptide linkage, an azo linkage and an aldehyde bond.

16. A method for synthesizing a chloroquine substance-coupled antibody composition comprising the steps of coupling;
a) a chloroquine substance to;
b) an active agent selected from the group consisting of protein active agents and peptide active agents.

17. The method of claim 16 wherein said coupling of chloroquine substance of (a) to said active agent of (b) includes a biocleavable linkage selected from the group consisting of an acid labile linkage, a disulfide linkage, a protected disulfide linkage, an ester linkage, an ortho ester linkage, a phosphonamide linkage, a biocleavable peptide linkage, an azo linkage and an aldehyde bond.

18. The method of claim 16 wherein said chloroquine substance of (a) is selected from the group consisting of quinoline compounds, 4-aminoquinoline compounds, 2-phenylquinoline compounds, chloroquines, hydroxychloroquines, amodiaquins, amopyroquines, halofantrines, mefloquines, nivaquines, primaquines, tafenoquines, quinone imines, chloroquine analogs or derivatives, (−)-enantiomers of chloroquine, (−)-enantiomers of hydroxychloroquine and amino, thio, phenyl, alkyl, vinyl and halogen derivatives thereof.

19. The method of claim 16 further comprising the step of coupling a targeting molecule to said chloroquine combinative agent.

20. The method of claim 16 further comprising the step of coupling a transduction vector to said chloroquine combinative agent.
